(19) United States
(12) Patent Application Publication
(10) Pub. No.: US 2010/0056627 A1
(43) Pub. Date: Mar. 4, 2010

(54) PHARMACEUTICAL COMPOSITIONS OF ALKYL GALLATES

(76) Inventors: Tomihiko Higuchi, Tokushima-ken (JP); Hirofumi Shibata, Tokushima-ken (JP); Masanori Higuchi, Tokushima-ken (JP)

Correspondence Address: WENDEROTH, LIND & PONACK, L.L.P., 1030 15th Street, N.W.,, Suite 400 East, Washington, DC, 20005-1503, US

(21) Appl. No.: 12/448,595

(22) PCT Filed: Dec. 27, 2007
(86) PCT No.: PCT/JP2007/075197

§ 371 Date: Jun. 26, 2009

(30) Foreign Application Priority Data
Dec. 28, 2006 (JP) 2006-356662
Oct. 12, 2007 (JP) 2007-267254
Dec. 14, 2007 (JP) 2007-323964

(51) Int. Cl. A61K 31/235; A61P 31/10; A61P 31/12; A61P 31/04

(52) U.S. Cl. 514/543

(57) ABSTRACT

It is intended to reinforce the anti-fungal, anti-viral and anti-bacterial activities of alkyl gallates, and to provide a new technology and means which allow the solubilization of the alkyl gallates in water.

A pharmaceutical composition of alkyl gallates of the present invention is characterized by containing (A) an alkyl gallate in which the carbon number of the alkyl group is in the range of 5 to 16 and (B) another alkyl gallate in which the carbon number of the alkyl group is smaller than that of (A). Preferably, the carbon number of the alkyl group of the alkyl gallate (B) is in the range of 2 to 7, and the composition further contains (C) at least one member selected from an alkali metal salt, boric acid, sodium borate and an organic salt.

### TECHNICAL FIELD

The present invention relates to a pharmaceutical composition having an anti-fungal, anti-bacterial, or anti-viral effect, which is useful as a pharmaceutical, an agrochemical, a cosmetic and a functional food product. More particularly, the invention relates to a method for reinforcing anti-fungal, anti-bacterial and anti-viral activities by alkyl gallates, and it relates to a new pharmaceutical composition useful as a therapeutic agent for infection or a prescribed drug for prevention in the fields of general external sterilization and disinfection, dermatology, oral dentistry (dental caries, periodontitis, halitosis, stomatitis), opthalmology, and gynecology (woman's health and sanitary protection) or as a pharmaceutical, an agrochemical (domestic animals, pets, marine life, plants), a cosmetic, a functional food product and the like.

### BACKGROUND ART

Some of alkyl gallates have been approved as food additives by WHO and FDA (propyl gallate, octyl gallate, dodecyl gallate) or as pharmaceutical additives (propyl gallate) or as quasi drugs (octyl gallate) by the Japanese Ministry of Health, Labour and Welfare; this means they are superior in safety.

The present inventors investigated these alkyl gallates from a new standpoint in detail and found that these have anti-fungal, anti-bacterial and anti-viral activities and proposed to use them as pharmaceuticals (Patent Document 1).

The anti-fungal, anti-bacterial and anti-viral activities of these alkyl gallates, however, are not always sufficiently strong, and reinforcement of their activities was desired. In addition, since the alkyl gallates were highly hydrophobic and sparingly soluble in water, their formulation was not always easy.

Patent document 1: JP-A-2006-306836

### DISCLOSURE OF INVENTION

### Problem to be Solved by the Invention

In the above-mentioned situation, the purpose of the invention is to reinforce the anti-fungal, anti-viral and anti-bacterial activities of the alkyl gallates by further developing and deepening the study by the inventors, and to provide a new technology and means which allow the solubilization of the alkyl gallates in water.

### Means for Solving the Problems

In order to solve the above-mentioned problem, the invention is characterized by the followings.

First: A pharmaceutical composition of alkyl gallates which contains alkyl gallates as active ingredients having an anti-fugal, anti-viral or anti-bacterial effect, in which the alkyl group of the alkyl gallate is bound to a galloyl group to form an ester linkage, characterized by comprising the following two members of alkyl gallates:

(A) an alkyl gallate in which the carbon number of the alkyl group is in the range of 5 to 16; and

(B) another alkyl gallate in which the carbon number of the alkyl group is smaller than that of (A).

Second: The first pharmaceutical composition of alkyl gallates, characterized in that the carbon number of the alkyl group of the alkyl gallate (B) is in the range of 2 to 7.
Third: The first or second pharmaceutical composition of alkyl gallates, characterized by further containing (C) at least one member selected from an alkali metal salt, boric acid, sodium borate and an organic salt.
Fourth: Any one of the first to third pharmaceutical compositions of alkyl gaellates, characterized in that it is an aqueous solution in which the alkyl gallates are solubilized by mixing the alkyl gallates with at least one member selected from a nonionic surfactant, polyethylene glycol and arginine or a hydrochloride of a derivative thereof in an aqueous solution or in a pH buffer.
Fifth: The fourth pharmaceutical composition of alkyl gallates, characterized in that it is an aqueous solution in which the alkyl gallates are solubilized by mixing 1 to 10 parts by weight of a nonionic surfactant and 100 to 5000 parts by weight of water based on 1 part by weight of alkyl gallates.
Sixth: The fifth pharmaceutical composition of alkyl gallates, characterized in that it is an aqueous solution in which the alkyl gallates are solubilized by mixing and heating the alkyl gallates at a temperature of 30 to 95° C. followed by cooling to room temperature.

### EFFECT OF THE INVENTION

According to the invention, the anti-fugal, anti-viral and anti-bacterial activities in the pharmaceutical containing alkyl gallates can be increased, and the alkyl gallates can be solubilized in water.

### BRIEF DESCRIPTION OF DRAWINGS

FIG. 1 is a graph showing shortening of the time required for killing bacteria by the concomitant use of octyl gallate and propyl gallate against MRSA COL strain.

FIG. 2 is a graph showing shortening of the time required for killing mold by the concomitant use of octyl gallate and propyl gallate against Candida albicans ATCC 10231.

FIG. 3 is a graph showing reinforcement of the influenza virucidal activity of n-dodecyl gallate by n-hexyl gallate and n-butyl gallate. The abscissa axis indicates the concentrations of n-dodecyl gallate.

FIG. 4 is a graph showing reinforcement of the anti-influenza viral activity of octyl gallate in MDCK cells by propyl gallate.

FIG. 5 is a graph showing the reinforcement effect of propyl gallate on the anti-viral activity of octyl gallate against HSV-1.

FIG. 6 is a graph showing shortening of the time required for killing viruses by the concomitant use of octyl gallate and propyl gallate against influenza B/T/1/05.

FIG. 7 is a graph showing the time course of the virucidal activity of octyl gallate alone against influenza virus B/T/1/05.

FIG. 8 is a graph showing the time course of the virucidal activity of propyl gallate alone against influenza virus B/T/1/05.

FIG. 9 is a graph showing the effect of a concentration of propyl gallate on the time course of the virucidal activity of octyl gallate (5 mg/L) against influenza virus B/T/1/05.

FIG. 10 is a graph showing the effect of a concentration of propyl gallate on the time course of the virucidal activity of octyl gallate (10 mg/L) against influenza virus B/T/0/05.

FIG. 11 is a graph showing the effect of a concentration of propyl gallate on the time course of the virucidal activity of octyl gallate (20 mg/L) against influenza virus B/T/1/05.

FIG. 12 is a graph showing the time course of reinforcement of the virucidal activity by the concomitant use of octyl gallate (30 mg/L) and propyl gallate (300 mg/L) against influenza virus B/T/1/05.

### BEST MODE FOR CARRYING OUT THE INVENTION

Hereinafter, an embodiment of the invention will be described.

The alkyl gallates used in the invention may have a proper other substituent or substituents, for example, an alkyl group, a cycloalkyl group, an aryl group, an alkoxy group, an ester group, an amide group, an amino group, or the like in addition to the ester linkage group between an alkyl group and a galloyl group.

Specific examples of the alkyl group of the alkyl gallates used in the invention include an n-propyl group, an isopropyl group, an n-butyl group, an isobutyl group, an n-amyl group, an isoamyl group, an n-hexyl group, an n-heptyl group, an n-octyl group, an n-nonyl group, an n-decyl group, an n-undecyl group, an n-dodecyl group and the like.

The terms “anti-fungal”, “anti-viral” and “anti-bacterial” in the invention also include the meanings of “fungicidal”, “virucidal” and “bactericidal”, respectively.

As described above, the pharmaceutical composition of alkyl gallates in which the effect of the active ingredient of the invention is reinforced is essentially characterized by comprising a plurality of:

(A) an alkyl gallate in which the carbon number of the alkyl group is in the range of 5 to 16; and

(B) another alkyl gallate in which the carbon number of the alkyl group is smaller than that of (A). Here, the carbon number of the alkyl group of the alkyl gallate (B) is preferably in the range of 2 to 7, and a combination of, for example, the alkyl gallate (A) in which the carbon number of the alkyl group is in the range of 8 to 12 and the alkyl gallate (B) in which the carbon number of the alkyl group is in the range of 3 to 7 is preferably exemplified.

The meaning of the above characteristic will be described in detail below.

In the following description, ND in the tables denotes “Not Detected”, which means the growth of bacteria is completely inhibited and no bacteria was detected.

### 1. Reinforcement of the Antimicrobial Activities

Table 1 shows that the MIC (minimum growth inhibition concentration) values of octyl gallate against gram-positive and gram-negative bacteria was decreased by the addition of isoamyl gallate at a concentration equal to or less than MIC at which no antimicrobial activity was observed, and that this decrease was further reinforced by the addition of NaCl.

It was found that similar phenomena were observed when n-heptyl gallate, n-hexyl gallate, n-pentyl gallate, n-butyl gallate, isobutyl gallate or n-propyl gallate (and their structurally similar type) was added instead of isoamyl gallate at a concentration equal to or less than MIC at which no antimicrobial activity was observed. It was also found that similarly, the MIC value of n-dodecyl gallate, n-undecyl gallate, n-decyl gallate, or n-nonyl gallate was decreased by the addition of n-heptyl gallate, n-hexyl gallate, n-pentyl gallate, n-butyl gallate, isobutyl gallate or n-propyl gallate (and their structurally similar type) at a concentration equal to or less than MIC at which no antimicrobial activity was observed.

Table 2 shows that the addition of isoamyl gallate at a concentration equal to or less than MIC at which no antimicrobial activity was observed and NaCl greatly decreased the MIC value of octyl gallate against bacteria, which was found to be far lower than that of an existing disinfectant chlorhexidine gluconate (a maximum of 640 times). This indicates that octyl gallate may be a potent bactericide or disinfectant.

The reason why the above-mentioned phenomena were caused is speculated as follows. By considering all the data available until now, the point of action of octyl gallate against bacteria includes 2 sites, i.e., the site involved in the growth inhibition of bacteria and the site not relating to the growth of bacteria. When isoamyl gallate once binds to the latter site, octyl gallate specifically binds only to the former site involved in the growth of bacteria; thus, it is speculated that octyl gallate inhibits the growth of bacteria at a far low concentration and decreases the MIC value.

On the basis of the above results, the invention will be summarized as follows.

The anti-fungal, anti-bacterial anti-viral activities of an alkyl gallate (A) (in which the carbon number of the alkyl chain is 5 to 16) is reinforced by another alkyl gallate (B) in which the carbon number of the alkyl chain is smaller than that of alkyl gallate (A) and an alkali metal salt such as a monovalent salt (C) (NaCl, KCl, LiCl, NaHCO₃), boric acid, sodium borate or an organic salt, and thus the MIC value of alkyl gallate (A) can be decreased.

TABLE 1

| | Effect of concomitant use of isoamyl gallate and NaCl on decrease of MIC | | | | | | | | | | |
|---|---|---|---|---|---|---|---|---|---|---|---|
| | of octyl gallate against a variety of bacteria | | | | | | | | | | |
| MIC sample | Octyl gallate MIC (μg/mL) | | | | | | | | | | |
| concomitant | NaCl | | | | | | | | | | |
| conc. (%) | none | 16 | 8 | 4 | | | | | | | |
| concomitant | Isoamyl gallate | | | | | | | | | | |
| conc. (μg/mL) | none | 25 | | none | 100 | 75 | 50 | 45 | 40 | 35 | 25 |
| time | 24 h | 24 h | 24 h | 24 h | 24 h | 24 h | 24 h | 24 h | 24 h | 24 h | 24 h |
| E. gallinarum (VanC1) | 62.5 | ND | ND | 31.25 | ND | ND | ND | ND | 0.24 | 0.49 | 7.81 |
| E. casseliflavus (VanC2/C3) | 62.5 | ND | ND | 62.50 | ND | 1.95 | 3.91 | ND | 3.91 | 3.91 | 15.63 |
| E. faecalis 0497P | 62.5 | ND | ND | 7.81 | ND | ND | ND | ND | ND | ND | ND |
| E. faecium 0677P | 62.5 | ND | ND | 31.25 | 0.98 | 7.81 | 15.63 | 7.81 | 15.63 | 15.63 | 15.63 |
| E. faecalis ATCC21212 | 62.5 | ND | ND | 7.81 | ND | ND | ND | ND | ND | ND | 3.91 |
| E. coli ATCC25922 | 62.5 | ND | ND | 0.98 | ND | ND | ND | ND | ND | ND | 31.25 |
| S. Typhimurium IFO13245 | 62.5 | ND | ND | ND | ND | ND | ND | ND | ND | ND | ND |
| S. epidermidis IFO37625 | 31.25 | ND | ND | 31.25 | ND | ND | 1.95 | ND | ND | 0.98 | 7.81 |
| S. epidermidis IID866 | 31.25 | ND | ND | 31.25 | ND | 0.49 | 7.81 | 1.95 | 3.91 | 7.81 | ND |
| S. marcescens IAM1184 | >250 | ND | ND | ND | ND | ND | ND | ND | ND | ND | ND |
| B. Subtilis IFO3134 | 31.25 | ND | ND | 15.63 | ND | ND | ≦0.061 | ND | 0.24 | 0.24 | 1.95 |
| | Effect of concomitant use of isoamyl gallate and NaCl on decrease of MIC | | | | | | | | | | |
| | of octyl gallate against a variety of bacteria | | | | | | | | | | |
| MIC sample | Octyl gallate MIC (μg/mL) | | | | | | | | | | |
| concomitant | NaCl | | | | | | | | | | |
| conc. (%) | 3 | | | | | | | 2 | | | |
| concomitant | Isoamyl gallate | | | | | | | | | | |
| conc. (μg/mL) | none | 100 | 75 | 50 | 45 | 40 | 35 | 100 | 75 | 50 | 25 |
| time | 24 h | 24 h | 24 h | 24 h | 24 h | 24 h | 24 h | 24 h | 24 h | 24 h | 24 h |
| E. gallinarum (VanC1) | 15.63 | 7.81 | 7.81 | 15.63 | 15.63 | 15.63 | 15.63 | 31.25 | 31.25 | 31.25 | 62.50 |
| E. casseliflavus (VanC2/C3) | 31.25 | 31.25 | 31.25 | 31.25 | 31.25 | 31.25 | 31.25 | 31.25 | 31.25 | 31.25 | 62.50 |
| E. faecalis 0497P | 31.25 | ND | 0.12 | 3.91 | 3.91 | 3.91 | 15.63 | 15.63 | 31.25 | 31.25 | 31.25 |
| E. faecium 0677P | 62.50 | 15.63 | 15.63 | 31.25 | 31.25 | 31.25 | 31.25 | 31.25 | 31.25 | 62.50 | 62.50 |
| E. faecalis ATCC21212 | 31.25 | 0.49 | 0.98 | 3.91 | 3.91 | 3.91 | 15.63 | 15.63 | 31.25 | 31.25 | 31.25 |
| E. coli ATCC25922 | 62.50 | 15.63 | 31.25 | 31.25 | 31.25 | 31.25 | 31.25 | 62.50 | 62.50 | 62.50 | 62.50 |
| S. Typhimurium IFO13245 | 31.25 | ND | ND | ND | ND | ND | 0.06 | ND | ND | 31.25 | 62.50 |
| S. epidermidis IFO37625 | 31.25 | ND | 0.98 | 7.81 | 7.81 | 7.81 | 15.63 | 1.95 | 7.81 | 15.63 | 15.63 |
| S. epidermidis IID866 | 31.25 | 0.49 | 3.91 | 7.81 | 7.81 | 15.63 | 15.63 | ND | ND | ND | 0.98 |
| S. marcescens IAM1184 | 0.12 | ND | ND | ND | ND | ND | ND | ND | >250 | >250 | >250 |
| B. Subtilis IFO3134 | 31.25 | ND | 0.12 | 3.91 | 3.91 | 3.91 | 3.91 | 0.49 | 1.95 | 3.91 | 15.63 |

TABLE 2
Comparison of MIC-values of octyl gallate and chlorhexidine gluconate
In this experiment, NaCl and isoamyl gallate were added at the time of determination of MIC of octyl gallate.
in Gram (+) and Gram (−) bacteria

| | chlorhexidine gluconate | octyl gallate | | Isoamyl gallate | A/B ratio |
|---|---|---|---|---|---|
| | MIC (μg/ml) (A) | MIC (μg/ml) (B) | NaCl % | μg/ml | fold |
| Gram (+) bacteria | | | | | |
| MRSA #5 | 0.61 | 0.06 | 4 | 25 | 10.2 |
| MRSA #9 | 1.22 | 0.06 | 4 | 25 | 20.3 |
| MRSA #17 | 1.22 | 0.06 | 4 | 50 | 20.3 |
| MRSA #22 | 1.22 | 0.06 | 4 | 50 | 20.3 |
| MRSA COL | 1.22 | 0.098 | 4 | 50 | 12.4 |
| MRSA Mu3 | 4.88 | 0.06 | 4 | 25 | 81.3 |
| MSSA 1023 | 0.61 | 0.06 | 4 | 25 | 10.2 |
| MSSA RN | 0.61 | 0.20 | 4 | 25 | 3.1 |
| E. faecium (VanA) | 1.20 | 0.10 | 2 | 100 | 12.0 |
| E. faecalis (VanB) | 4.90 | 0.49 | 4 | 40 | 10.0 |
| E. gallinarum (VanC1) | 4.90 | 0.24 | 4 | 40 | 20.4 |
| E. casseliflavus (VanC2/C3) | 4.90 | 1.95 | 4 | 75 | 2.5 |
| E. faecalis 0497P | 2.40 | 0.122 | 3 | 75 | 19.7 |
| E. faecium 0677P | 1.20 | 0.98 | 4 | 100 | 1.2 |
| E. faecalis ATCC21212 | 4.90 | 0.49 | 3 | 75 | 10.0 |
| S. epidermidis IFO3762 | 0.60 | 0.98 | 3 | 75 | 0.6 |
| S. epidermdis IID866 | 0.60 | 0.49 | 4 | 75 | 1.2 |
| B. subtilis IFO3134 | 1.20 | 0.122 | 4 | 75 | 9.8 |
| Gram (−) bacteria | | | | | |
| S. Typhimurium IFO13245 | 4.90 | 0.06 | 3 | 35 | 81.7 |
| P. aeruginosa ATCC9027 | 19.50 | 1.95 | 3 | 20 | 10.0 |
| P. aeruginosa PAO1 | 78.10 | 0.122 | 3 | 25 | 640.2 |
| E. coli ATCC25922 | 2.40 | 0.98 | 4 | 0 | 2.4 |
| S. marcescens IAM1184 | 9.80 | 0.122 | 3 | 0 | 80.3 |

In this connection, the experimental procedures in the cases of Tables 1 and 2 as well as in Table 8 shown below are as follows.

Determination of the minimum growth inhibition concentration (MIC) was conducted according to the standard method of the Japanese Society of Chemotherapy (Susumu Mitsuhashi et al., 1981; Revision of the method for determination of the minimum growth inhibition concentration (MIC), Chemotherapy, 29:76-79) on a Mueller Hinton II Agar (BBL) by a 2-fold serial dilution method using an agar plate. The reagents employed are octyl gallate (Tokyo Kasei), isoamyl gallate (Tokyo Kasei), and NaCl (Kanto Chemical Co.). A 5% Hibitane solution (Sumitomo Seiyaku) was used as a reference drug. The test organism was inoculated into a Mueller Hinton Broth (DIFCO), cultured at 37° C. for 18 hours for multiplication, and diluted with physiological saline to 1×10⁶ CFU/mL to give a cell solution for inoculation. This cell solution was inoculated with a microplanter (Sakuma Seisakusho) on an agar plate supplemented with a drug. After incubation at 37° C. for 24 hours, the concentration at which the growth was completely inhibited was regarded as MIC (minimum inhibition concentration).

Table 3A shows the reinforcement effect of addition of 0.9% (w/v) trisodium citrate and propyl gallate on the antimicrobial activity of octyl gallate against common bacteria (gram-positive and gram-negative bacteria).

It is clearly shown that the antimicrobial activity of octyl gallate against common bacteria (gram-positive and gram-negative bacteria) is reinforced by the addition of 0.9% (w/v) trisodium citrate and propyl gallate. In this connection, as the amount of trisodium citrate added increased, the MIC value of octyl gallate decreased by propyl gallate at a lower concentration. Also in the case where disodium hydrogen citrate was used instead of trisodium citrate, a similar tendency was observed. In the table, MHA denotes Mueller Hinton II Agar, and DDW denotes sterile water. Octyl gallate was solubilized with J1816 (in an amount three times larger than that of octyl gallate) by the method in Example 6 mentioned below. Propyl gallate was solubilized by the method in Example 5 mentioned below.

TABLE 3A

| Date | Sep. 6, 2007 | | | | | | | | | | | |
|---|---|---|---|---|---|---|---|---|---|---|---|---|
| MIC sample | Octyl gallate MIC (μg/mL) | | | | | | Octyl gallate MIC (μg/mL) | | | | | |
| medium | MHA | | | | | | MHA (+0.9% trisodium citrate) | | | | | |
| concomitant | DDW | Propyl gallate | | | | | DDW | Propyl gallate | | | | |
| conc. (μg/mL) | | 400 | 350 | 300 | 250 | 200 | | 400 | 350 | 300 | 250 | 200 |
| time | 24 h | 24 h | 24 h | 24 h | 24 h | 24 h | 24 h | 24 h | 24 h | 24 h | 24 h | 24 h |
| E. faecium (VanA) | 50 | ND | ND | ND | ND | ND | 50 | ND | ND | ND | ND | ND |
| E. faecalis (VanB) | 50 | ND | ND | ND | 20 | 20 | 50 | 2.5 | 10 | 20 | 20 | 20 |
| E. gallinarum (VanC1) | 50 | 2.5 | 10 | 20 | 20 | 20 | 50 | ND | 5 | 10 | 10 | 20 |
| E. casseliflavus (VanC2/C3) | 50 | 10 | 20 | 20 | 50 | 50 | 50 | 0.3125 | 5 | 20 | 20 | 20 |
| E. faecalis 0497P | 50 | ND | ND | ND | ND | 1.25 | 50 | ND | 5 | 10 | 20 | 20 |
| E. faecium 0677P | 50 | ND | ND | ND | 20 | 20 | 50 | ND | ND | 5 | 10 | 20 |
| E. faecalis ATCC21212 | 50 | ND | ND | ND | 20 | 20 | 50 | ND | 10 | 10 | 10 | 20 |
| E. coli ATCC25922 | 50 | ND | ND | ND | 20 | 20 | 50 | ND | 10 | 10 | 20 | 20 |
| E. coli KUE050701 | >50 | ND | ND | ND | >50 | >50 | >50 | ND | ND | ND | ND | ND |
| S. Typhimurium IFO13245 | 50 | ND | ND | ND | ND | ND | 50 | ND | ND | ND | ND | ND |
| S. Enterietidis IFO63313 | >50 | ND | ND | ND | ND | ND | >50 | ND | ND | ND | ND | ND |
| S. Enterietidis DT104-3 | >50 | ND | ND | ND | ND | ND | >50 | ND | ND | ND | ND | ND |
| S. Enterielidis DT104-26 | >50 | ND | ND | ND | ND | ND | >50 | ND | ND | ND | ND | ND |
| S. Oranienburg 1151 | >50 | ND | ND | ND | ND | ND | >50 | ND | ND | ND | ND | ND |
| S. Infantis TUS050902 | >50 | ND | ND | ND | ND | ND | >50 | ND | ND | ND | ND | ND |
| P. aeruginosa ATCC9027 | >50 | >50 | >50 | >50 | >50 | >50 | >50 | ND | ND | ND | ND | >50 |
| P. aeruginosa PAO1 | >50 | ND | ND | ND | ND | >50 | >50 | ND | ND | ND | ND | ND |
| P. aeruginosa MHP0509-01 | >50 | ND | ND | ND | >50 | >50 | >50 | ND | ND | ND | ND | 20 |
| K. pneumoniae ATCC10031 | >50 | >50 | >50 | >50 | >50 | >50 | >50 | ND | ND | >50 | >50 | >50 |
| K. pneumoniae KUK050801 | >50 | >50 | >50 | >50 | >50 | >50 | >50 | >50 | >50 | >50 | >50 | >50 |
| S. epidermidis IFO3762 | 20 | ND | ND | ND | ND | 5 | 20 | ND | ND | ND | ND | ND |
| S. epidermidis IID866 | 20 | ND | ND | ND | ND | ND | 20 | ND | ND | ND | ND | ND |
| S. marcescens IAM1184 | >50 | ND | ND | ND | ND | >50 | >50 | ND | ND | ND | ND | ND |
| B. subtilis IFO3134 | 50 | ND | 1.25 | 5 | 10 | 10 | 20 | ND | ND | 2.5 | 5 | 10 |
| P. mirabilis IFO3849 | >50 | ND | ND | ND | ND | ND | >50 | ND | ND | ND | ND | ND |
| F. cloacae IFO13535 | >50 | ND | ND | ND | ND | ND | >50 | ND | ND | ND | ND | ND |
| MRSA COL | 25 | ND | ND | ND | ND | ND | 20 | ND | ND | ND | ND | ND |

Table 3B shows the MIC values of octyl gallate (solubilized with J1216 by the method in Example 2 mentioned below) obtained by recrystallization so as to have a high purity against clinical isolates MRSA (21 strains) and MSSA (8 strains). This reveals that disodium hydrogen citrate reinforces the antimicrobial activity of octyl gallate more than trisodium citrate, and that by the concomitant use with 50 mg/L of propyl gallate (solubilized by the method in Example 3 mentioned below), the antimicrobial activity of octyl gallate is significantly reinforced and all strains were killed with 1.25 mg/L of octyl gallate. From the results of this experiment, it was demonstrated that not the impurities of octyl gallate show an antimicrobial activity, but octyl gallate per se shows an antimicrobial activity.

Table 3C shows the MIC values of octyl gallate (solubilized with J1216 by the method in Example 2 mentioned below) obtained by recrystallization so as to have a high purity against gram-positive and gram-negative bacteria. This reveals that disodium hydrogen citrate reinforces the antimicrobial activity of octyl gallate more than trisodium citrate (the right column in the table), and that by the concomitant use with 300 mg/L of propyl gallate, the bacteria, except for three strains of bacteria, were completely killed. Further, it is found that by the concomitant use with 100 mg/L of propyl gallate, the MIC value of octyl gallate can be decreased (the antimicrobial activity of octyl gallate can be reinforced). From the results of this experiment, it was demonstrated that not the impurities of octyl gallate show an antimicrobial activity, but octyl gallate per se shows an antimicrobial activity against common bacteria.

TABLE 3B

| Date | Dec. 24, 2007 | | | Dec. 7, 2007 | | | |
|---|---|---|---|---|---|---|---|
| MIC sample | #Recrystalized octyl gallete MIC (mg/L) | | | #Recrystalized octyl gallate MIC(mg/L) | | | |
| medium | MHA (+0.9% disodium hydrogen citrate)* | | | MHA (+0.9% trisodium citrate)* | | | |
| | | Propyl gallate (dissolved at 5 mg/mL in | | | Propyl gallate (dissolved at 5 mg/mL in water | | |
| concomitant | DDW | water) | | J-1216 | containing 3 mg/mL J-1216) | | |
| conc. (mg/L) | | 100 | 50 | 600 | 200 | 100 | 75 |
| time | 24 h | 24 h | 24 h | 24 h | 24 h | 24 h | 24 h |
| MRSA #1 | 0.625 | ND | ND | 25 | 2.5 | ND | 10 |
| MRSA #2 | 2.5 | ND | ND | 50 | 2.5 | 2.5 | 10 |
| MRSA #3 | ND | ND | ND | 20 | 2.5 | ND | 10 |
| MRSA #4 | ND | ND | ND | ND | ND | ND | ND |
| MRSA #5 | ND | ND | ND | 20 | ND | ND | 5 |
| MRSA #6 | ND | ND | ND | 20 | ND | ND | 5 |
| MRSA #7 | ND | ND | ND | 20 | ND | ND | 2.5 |
| MRSA #8 | ND | ND | ND | 20 | ND | ND | 5 |
| MRSA #9 | ND | ND | ND | 20 | ND | ND | 5 |
| MRSA #10 | ND | ND | 1.25 | 25 | 0.625 | ND | 10 |
| MRSA #12 | ND | ND | ND | 50 | 5 | ND | 10 |
| MRSA #13 | ND | ND | ND | 20 | ND | ND | 5 |
| MRSA #16 | 1.25 | ND | ND | 50 | ND | ND | 10 |
| MRSA #17 | ND | ND | ND | 50 | ND | ND | 10 |
| MRSA #18 | ND | ND | ND | ND | ND | ND | ND |
| MRSA #19 | ND | ND | ND | 20 | ND | ND | 5 |
| MRSA #20 | ND | ND | ND | 20 | 5 | ND | 10 |
| MRSA #21 | ND | ND | ND | 25 | 2.5 | 5 | 10 |
| MRSA #22 | 2.5 | ND | 0.625 | 50 | 2.5 | 5 | 10 |
| MRSA COL | 2.5 | ND | 1.25 | 20 | 1.25 | 1.25 | 5 |
| MRSA Mu3 | ND | ND | ND | 25 | ND | ND | 10 |
| MSSA 1003 | ND | ND | 0.625 | ND | 1.25 | ND | 5 |
| MSSA 1010 | ND | ND | ND | ND | 5 | ND | 10 |
| MSSA 1020 | ND | ND | ND | 5 | ND | ND | 5 |
| MSSA 1023 | 2.5 | ND | ND | 20 | 2.5 | 5 | 10 |
| MSSA 1029 | 2.5 | ND | ND | 25 | 2.5 | 5 | 10 |
| MSSA 1032 | ND | ND | ND | 25 | ND | ND | 10 |
| MSSA ATCC6538 | ND | ND | ND | ND | ND | ND | 5 |
| MSSA RN4220 | ND | ND | ND | ND | ND | ND | 2.5 |
| | 24 h MIC | 24 h MIC | 24 h MIC | 24 h MIC | 24 h MIC | 24 h MIC | 24 h MIC |
| | MRSA | MRSA | MRSA | MRSA | MRSA | MRSA | MRSA |
| MIC Range | 0.625-2.5 | ND | 0.625-1.25 | 20-50 | 0.0625-5 | 1.25-5 | 2.5-10 |
| MIC₅₀ | ND | ND | ND | 20 | 2.5 | 2.5 | 10 |
| MIC₁₀₀ | 2.5 | ND | 1.25 | 50 | 5 | 5 | 10 |

TABLE 3C

| Date | Dec. 24, 2007 | | | Jul. 25, 2007 | | | Sep. 6, 2007 | | | Nov. 21, 2007 | | | |
|---|---|---|---|---|---|---|---|---|---|---|---|---|---|
| | | | | Octyl gallate (was solubilized | | | Octyl gallate | | | | | | |
| | | | | with 1 mM phosphate | | | (dissolved in 1 mM | | | Octyl gallate (dissolved | | | |
| | | | | buffer at pH 6.5 containing | | | phosphate buffer | | | at 1 mg/mL in MilliQ | | | |
| | #Recrystalized octyl | | | 0.356 mg/mL of J-1816) | | | involving 3 mg/mL | | | water containing 3 mg/mL | | | |
| MIC sample | gallate MIC (mg/L) | | | MIC (mg/L) | | | J-1816) MIC (mg/L) | | | of J-1216) MIC (mg/L) | | | |
| | MHA (+0.9% disodium | | | MHA (+0.9% | | | MHA (+0.9% | | | MHA (+0.9% | | | |
| medium | hydrogen citrate)* | | | trisodium citrate) | | | trisodium citrate) | | | trisodium citrate)* | | | |
| | | | | 1 mM | | | | | | | | | |
| | | | | phosphate | | | | | | | | | |
| | | | | buffer at | Propyl gallate | | | Propyl gallate | | | | | |
| | | | | pH 6.5 | (dissolved in 1 mM | | | (dissolved in | | | | | |
| | | Propyl gallate | | involving | phosphate buffer at | | | milliQ water | | | Propyl gallate | | |
| | | (dissolved at | | 0.356 | pH 6.5 containing | | | containing | | | (dissolved | | |
| | | 5 mg/mL in | | mg/mL | 0.356 mg/mL | | | 1.5 mg/mL | | | at 3 mg/mL | | |
| concomitant | DDW | milliQ water) | | J-1816 | J-1816) | | DDW | of J-1816) | | DDW | in milliQ water) | | |
| conc. (mg/L) | | 300 | 100 | 0.1 mM | 200 | 100 | | 300 | 200 | | 300 | 200 | 100 |
| time | 24 h | 24 h | 24 h | 24 h | 24 h | 24 h | 24 h | 24 h | 24 h | 24 h | 24 h | 24 h | 24 h |
| E. faecium (VanA) | ND | ND | ND | 50 | 10 | 50 | 50 | ND | ND | 20 | ND | 10 | 20 |
| E. faecalis (VanB) | 10 | ND | 10 | 50 | 50 | 50 | 50 | 20 | 20 | 20 | 20 | 20 | 20 |
| E. gallinarum (VanC1) | 10 | ND | 5 | 50 | 50 | 50 | 50 | 10 | 20 | 20 | 20 | 20 | 20 |
| E. casseliflavus (VanC2/C3) | 10 | ND | 5 | 50 | 50 | 50 | 50 | 20 | 20 | 20 | 20 | 20 | 20 |
| E. faecalis 0497P | 10 | ND | 5 | 50 | 50 | 50 | 50 | 10 | 20 | 20 | 20 | 20 | 20 |
| E. faecium 0677P | 10 | ND | 5 | 50 | 50 | 50 | 50 | 5 | 20 | 20 | 20 | 20 | 20 |
| E. faecalis ATCC21212 | 10 | ND | 5 | 50 | 50 | 50 | 50 | 10 | 20 | 20 | 20 | 20 | 20 |
| E. coli ATCC25922 | 50 | ND | ND | 50 | 50 | 50 | 50 | 10 | 20 | 20 | ND | ND | 50 |
| E. coli KUE050701 | 50 | ND | ND | >100 | 25 | >100 | >100 | ND | ND | 50 | ND | 10 | >100 |
| S. Typhimurium IFO13245 | 50 | ND | ND | 50 | ND | 2.5 | 50 | ND | ND | 20 | ND | ND | 10 |
| S. Enterietidis IFO63313 | 50 | ND | ND | 50 | ND | 10 | >100 | ND | ND | 50 | ND | ND | 5 |
| S. Enterietidis DT104-3 | 100 | ND | ND | >100 | ND | 50 | >100 | ND | ND | 100 | ND | ND | 50 |
| S. Enterietidis DT104-26 | 100 | ND | ND | 100 | ND | 50 | >100 | ND | ND | 100 | ND | ND | 20 |
| S. Oranienburg 1151 | 100 | ND | ND | >100 | ND | 50 | >100 | ND | ND | >100 | ND | ND | ND |
| S. Infantis TUS050902 | 25 | ND | ND | >100 | ND | ND | >100 | ND | ND | 20 | ND | ND | ND |
| P. aeruginosa ATCC9027 | 100 | ND | ND | >100 | 20 | >100 | >100 | ND | 100 | 100 | ND | 25 | >100 |
| P. aeruginosa PAO1 | >100 | ND | ND | >100 | ND | 100 | >100 | ND | ND | 100 | ND | 25 | >100 |
| P. aeruginosa MHP0509-01 | >100 | ND | ND | >100 | 100 | >100 | >100 | ND | 20 | 100 | 25 | >100 | >100 |
| K. pneumoniae ATCC10031 | >100 | >100 | >100 | >100 | >100 | >100 | >100 | 100 | >100 | >100 | >100 | >100 | >100 |
| K. pneumoniae KUK050801 | >100 | >100 | >100 | >100 | >100 | >100 | >100 | >100 | >100 | >100 | >100 | >100 | >100 |
| S. epidermidis IFO3762 | ND | ND | ND | 20 | 5 | 10 | 20 | ND | ND | 10 | 0.625 | 5 | 10 |
| S. epidermidis IID866 | ND | ND | ND | 10 | ND | ND | 20 | ND | ND | ND | 5 | 5 | 10 |
| S. marcescens IAM1184 | >100 | >100 | >100 | >100 | 50 | >100 | >100 | ND | ND | 100 | >100 | >100 | >100 |
| B. subtilis IFO3134 | 5 | ND | 5 | 25 | 10 | 20 | 20 | 2.5 | 10 | 10 | 2.5 | 5 | 5 |
| P. mirabilis IFO3849 | >100 | ND | ND | >100 | ND | 25 | 100 | ND | ND | >100 | >100 | >100 | >100 |
| E. cloacae IFO13535 | >100 | ND | ND | >100 | ND | 50 | >100 | ND | ND | >100 | ND | ND | 50 |
| MRSA COL | 5 | ND | ND | 50 | ND | ND | 20 | ND | ND | 10 | ND | 1.25 | 5 |

MHA, Muller Hinton Agar
DDW, [symbol] * Sodium ascorbate was added at 0.5 mg/ml in the medium (Table 3B-3C).
#Recrystalized octyl gallate was dispersed at 1 mg/mL in hot water at 70 C. by vigorous shaking and solubilized by adding 3 mg/mL of J-1216.

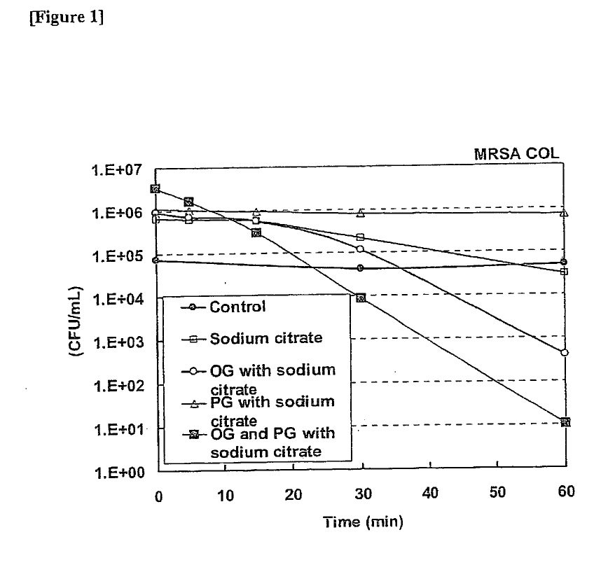

FIG. 1 shows shortening of the time required for killing bacteria by the concomitant use of octyl gallate and propyl gallate against MRSA COL strain. It is found that the bactericidal activity of propyl gallate alone is low, however, by the concomitant use with octyl gallate, the time required for killing bacteria is shortened to a large extent.

Table 4 shows the effect of addition of NaCl on the antimicrobial activity of octyl gallate against common bacteria (gram-positive and gram-negative bacteria).

It is found that as the amount of NaCl added (2 to 4%) increases, the MIC value of octyl gallate is markedly decreased by isoamyl gallate.

TABLE 4

| Date | Nov. 17, 2006 & Dec. 1, 2006 | | | | | | | | | | | |
|---|---|---|---|---|---|---|---|---|---|---|---|---|
| MIC sample | Octyl gallete MIC (μg/mL) | | | | | | | | | | | |
| concomitant | NaCl | | | | | | | | | | | |
| conc. (%) | none | 16 | 8 | 4 | | | | | | | | 3 |
| concomitant | Isoamyl gallate | | | | | | | | | | | |
| conc. (μg/mL) | none | 25 | | none | 100 | 75 | 50 | 45 | 40 | 35 | 25 | none |
| time | 48 h | 48 h | 48 h | 48 h | 48 h | 48 h | 48 h | 48 h | 48 h | 48 h | 48 h | 48 h |
| E. faecium (VanA) | 125 | ND | ND | ND | 7.8125 | ND | ND | ND | ND | ND | ND | 15.625 |
| E. faecalis (VanB) | 125 | ND | ND | 15.625 | 31.25 | 3.906 | 7.8125 | 15.625 | 15.625 | 15.625 | 15.625 | 62.5 |
| E. gallinarum (VanC1) | 125 | ND | ND | 7.8125 | 31.25 | 7.8125 | 7.8125 | 3.906 | 7.8125 | 15.625 | 15.625 | 62.5 |
| E. cosseliflavus | 125 | ND | ND | 15.625 | 62.5 | 7.8125 | 7.8125 | 3.906 | 15.625 | 15.625 | 15.625 | 62.5 |
| (VanC2/C3) | | | | | | | | | | | | |
| E. faecalis 0497P | 125 | ND | ND | 0.1221 | 15.625 | ND | ND | ND | ND | ND | ND | 31.25 |
| E. faecium 0677P | 125 | ND | ND | 15.625 | 62.5 | 15.625 | 15.625 | 15.625 | 15.625 | 15.625 | 31.25 | 62.5 |
| E. faecalis ATCC21212 | 125 | ND | ND | 3.906 | 15.625 | ND | ND | 0.2441 | ≦0.061 | 0.2441 | 1.9531 | 31.25 |
| E. coli ATCC25922 | 62.5 | ND | ND | 31.25 | 62.5 | ND | ND | 15.625 | 31.25 | 15.625 | 15.625 | 125 |
| E. coli KUE050701 | 125 | ND | ND | 250 | >250 | 125 | 62.5 | >250 | >250 | >250 | >250 | >250 |
| S. Typhimurium IFO13245 | 62.5 | ND | ND | ND | 15.625 | ND | ND | ND | ND | ND | ND | 62.5 |
| S. Enterietidis IFO63313 | 250 | ND | ND | 250 | >250 | ND | 62.5 | >250 | >250 | >250 | >250 | >250 |
| S. Enterietidis DT104-3 | 125 | ND | ND | 125 | 250 | ND | ND | 62.5 | 62.5 | 62.5 | >250 | 250 |
| S. Enterietidis DT104-26 | 62.5 | ND | ND | 62.5 | 125 | ND | ND | 62.5 | 62.5 | 62.5 | 250 | 125 |
| S. oranienburg 1151 | 125 | ND | ND | 125 | 250 | ND | ND | 15.626 | 31.25 | 31.25 | >250 | 250 |
| S. infantis TUSD50902 | 125 | ND | ND | 62.5 | 62.5 | ND | ND | 31.25 | 31.25 | 31.25 | 62.5 | 125 |
| P. aeruginosa ATCC9027 | 125 | ND | ND | ND | ND | ND | ND | ND | ND | ND | ND | 62.5 |
| P. aeruginosa PAO1 | 250 | ND | ND | ND | 31.25 | ND | ND | ND | ND | ND | ND | 125 |
| P. aeruginosa | >250 | ND | ND | ND | ND | ND | ND | ND | ND | ND | ND | ND |
| MHP0509-01 | | | | | | | | | | | | |
| K. pnemoniae ATCC10031 | 125 | ND | ND | 62.5 | 62.5 | ND | ND | 31.25 | 62.5 | 31.25 | 62.5 | 250 |
| K. pnemoniae KUK050801 | 250 | ND | ND | 250 | 125 | ND | 62.5 | 62.5 | 62.5 | 62.5 | >250 | >250 |
| S. epidermidis IFO3762 | 62.5 | ND | 3.906 | 31.25 | 31.25 | ND | 1.953 | 15.625 | 15.625 | 15.625 | 15.625 | 62.5 |
| S. epidermidis IID866 | 62.5 | ND | ND | 3.906 | 31.25 | 1.9531 | 3.906 | 15.625 | 15.625 | 31.25 | 31.25 | 62.5 |
| S. marcescens IAM1184 | >250 | ND | ND | ND | 62.5 | ND | ND | ND | ND | ND | ND | 250 |
| B. subtilis IFO3134 | 31.25 | ND | ND | 7.8125 | 31.25 | ≦0.061 | 3.906 | 1.953 | 7.8125 | 15.625 | 15.625 | 31.25 |
| P. mirabilis IFO3849 | >250 | ND | ND | 250 | >250 | ND | ND | >250 | >250 | >250 | >250 | >250 |
| E. cloacae IFO13535 | >250 | ND | ND | 125 | >250 | ND | ND | 62.5 | 62.5 | 62.5 | >250 | >250 |
| A. calcorceticus | 31.25 | ND | ND | ND | 15.625 | ND | ND | ND | ND | ND | ND | 31.25 |
| ATCC19606 | | | | | | | | | | | | |

| Date | Nov. 17, 2006 & Dec. 1, 2006 | | | | | | | | | |
|---|---|---|---|---|---|---|---|---|---|---|
| MIC sample | Octyl gallete MIC (μg/mL) | | | | | | | | | |
| concomitant | NaCl | | | | | | | | | |
| conc. (%) | 3 | | | | | | 2 | | | |
| concomitant | Isoamyl gallate | | | | | | | | | |
| conc. (μg/mL) | 100 | 75 | 50 | 45 | 40 | 35 | 100 | 75 | 50 | 25 |
| time | 48 h | 48 h | 48 h | 48 h | 48 h | 48 h | 48 h | 48 h | 48 h | 48 h |
| E. faecium (VanA) | ND | ND | ND | ND | ND | ND | 0.1221 | 0.2441 | 7.8125 | 3.906 |
| E. faecalis (VanB) | 31.25 | 31.25 | 31.25 | 31.25 | 31.25 | 31.25 | 62.5 | 62.5 | 62.5 | 62.5 |
| E. gallinarum (VanC1) | 31.25 | 31.25 | 31.25 | 31.25 | 31.25 | 31.25 | 62.5 | 62.5 | 62.5 | 62.5 |
| E. cosseliflavus | 31.25 | 31.25 | 31.25 | 31.25 | 31.25 | 31.25 | 62.5 | 62.5 | 62.5 | 62.5 |
| (VanC2/C3) | | | | | | | | | | |
| E. faecalis 0497P | 0.2441 | 3.906 | 7.8125 | 7.8125 | 15.625 | 15.625 | 31.25 | 31.25 | 62.5 | 62.5 |
| E. faecium 0677P | 31.25 | 31.25 | 31.25 | 62.5 | 31.25 | 31.25 | 62.5 | 125 | 125 | 125 |
| E. faecalis ATCC21212 | 7.8125 | 7.8125 | 7.8125 | 7.8125 | 15.625 | 15.625 | 62.5 | 62.5 | 62.5 | 31.25 |
| E. coli ATCC25922 | 62.5 | 62.5 | 62.5 | 62.5 | 62.5 | 62.5 | 62.5 | 62.5 | 125 | 125 |
| E. coli KUE050701 | >250 | >250 | >250 | >250 | >250 | >250 | >250 | >250 | >250 | >250 |
| S. Typhimurium IFO13245 | ND | ND | 3.906 | 7.8125 | 31.25 | 15.625 | 15.625 | 62.5 | 62.5 | 62.5 |
| S. Enterietidis IFO63313 | 62.5 | 125 | >250 | >250 | >250 | >250 | 62.5 | >250 | >250 | >250 |
| S. Enterietidis DT104-3 | 62.5 | 62.5 | 125 | 125 | 125 | 250 | 62.5 | 250 | 250 | 250 |
| S. Enterietidis DT104-26 | 62.5 | 62.5 | 125 | 125 | 125 | 125 | 62.5 | 125 | 125 | 125 |
| S. oranienburg 1151 | ND | 31.25 | 125 | 250 | 250 | 250 | 62.5 | 125 | 250 | 250 |
| S. infantis TUSD50902 | 62.5 | 62.5 | 62.5 | 62.5 | 62.5 | 62.5 | 62.5 | 62.5 | 125 | 125 |
| P. aeruginosa ATCC9027 | ND | ND | 15.625 | 31.25 | 31.25 | 15.625 | ND | 62.5 | 62.5 | 125 |
| P. aeruginosa PAO1 | ND | ND | 62.5 | 62.5 | 125 | 62.5 | 31.25 | 62.5 | >250 | 250 |
| P. aeruginosa | ND | ND | ND | ND | ND | ND | 125 | >250 | >250 | 250 |
| MHP0509-01 | | | | | | | | | | |
| K. pnemoniae ATCC10031 | 62.5 | 125 | 125 | 125 | 125 | 125 | 250 | 250 | 250 | 250 |
| K. pnemoniae KUK050801 | >250 | >250 | >250 | >250 | 125 | >250 | >250 | >250 | >250 | >250 |
| S. epidermidis IFO3762 | ND | 15.625 | 31.25 | 31.25 | 31.25 | 31.25 | 31.25 | 31.25 | 31.25 | 31.25 |
| S. epidermidis IID866 | 3.906 | 15.625 | 31.25 | 31.25 | 31.25 | 31.25 | ND | 31.25 | 31.25 | 31.25 |
| S. marcescens IAM1184 | ND | 250 | 250 | 250 | 250 | 125 | >250 | >250 | >250 | >250 |
| B. subtilis IFO3134 | 3.906 | 7.8125 | 15.625 | 15.625 | 15.625 | 15.625 | 7.8125 | 3.906 | 7.8125 | 15.625 |
| P. mirabilis IFO3849 | ND | >250 | >250 | >250 | >250 | >250 | >250 | >250 | >250 | >250 |
| E. cloacae IFO13535 | 62.5 | 62.5 | 125 | >250 | 250 | >250 | 125 | >250 | >250 | >250 |
| A. calcorceticus | ND | ND | ND | ND | ND | ND | ND | ND | ND | ND |
| ATCC19606 | | | | | | | | | | |

Table 5 shows the reinforcement effect of addition of 0.9% (w/v) trisodium citrate and propyl gallate on the antimicrobial activity of octyl gallate against fungi (mold).

It is clearly shown that the antimicrobial activity of octyl gallate against fungi (mold) is reinforced by the addition of 0.9% (w/v) trisodium citrate and propyl gallate.

TABLE 5

| Date | 2007.7.25 | | | | | | | | | | | | | | | | | |
|---|---|---|---|---|---|---|---|---|---|---|---|---|---|---|---|---|---|---|
| MIC sample | #1Octyl gallate MIC (μg/mL) | | | | | | | | | | #1 Octyl gallate MIC (μg/mL) | | | | | | | |
| Medium | SDA | | | | | | | | | | SDA (+0.9% trisodium citrate) | | | | | | | |
| | phosphate | | | | | | | | | | phosphate | | | | | | | |
| Concomitant | buffer (1 mM) | | #2 Propyl gallate | | | | | | | | buffer (1 mM) | | #2 Propyl gallate | | | | | |
| Conc. (μg/mL) | 0 | | 150 | | 300 | | 500 | | 1000 | | 0 | | 150 | | 300 | | 1000 | |
| Time | 24 hr | 48 hr | 24 hr | 48 hr | 24 hr | 48 hr | 24 hr | 48 hr | 24 hr | 48 hr | 24 hr | 48 hr | 24 hr | 48 hr | 24 hr | 48 hr | 24 hr | 48 hr |
| C. albicans | 20 | 20 | 20 | 20 | 20 | 20 | 20 | 20 | 10 | 10 | 20 | 20 | 20 | 20 | 10 | 20 | 10 | 10 |
| ATCC10231 | 20 | 20 | 20 | 20 | 20 | 20 | 10 | 10 | 10 | 10 | 20 | 20 | 20 | 20 | 10 | 10 | 10 | 10 |
| | 20 | 20 | 20 | 20 | 20 | 20 | 20 | 20 | 10 | 10 | 20 | 20 | 20 | 20 | 10 | 20 | 10 | 10 |
| Candida spp. | 50 | 50 | 50 | 50 | 50 | 50 | 50 | 50 | 50 | 50 | 20 | 50 | 20 | 25 | 10 | 20 | 10 | 20 |
| | 50 | 50 | 50 | 50 | 50 | 50 | 50 | 50 | 50 | 50 | 20 | 50 | 20 | 25 | 10 | 20 | 10 | 20 |
| | 50 | 50 | 50 | 50 | 50 | 50 | 50 | 50 | 50 | 50 | 20 | 50 | 20 | 25 | 10 | 20 | 10 | 20 |
| S. cerevisiae | 20 | 20 | 20 | 20 | 20 | 20 | 20 | 20 | 10 | 10 | 10 | 20 | 10 | 20 | 10 | 20 | 5 | 10 |
| ATCC9763 | 20 | 20 | 20 | 20 | 20 | 20 | 20 | 20 | 10 | 10 | 10 | 20 | 10 | 20 | 10 | 20 | 5 | 10 |
| | 20 | 20 | 20 | 20 | 20 | 20 | 20 | 20 | 10 | 10 | 10 | 20 | 10 | 20 | 10 | 20 | 5 | 10 |
| Yeast 4-1 white | 10 | 10 | 10 | 10 | 10 | 10 | 10 | 10 | 10 | 10 | 10 | 10 | 10 | 10 | 10 | 10 | 5 | 5 |
| | 10 | 10 | 5 | 5 | 5 | 5 | 10 | 10 | 5 | 5 | 10 | 10 | 5 | 5 | 5 | 5 | 5 | 5 |
| | 10 | 10 | 5 | 5 | 10 | 10 | 5 | 5 | 5 | 5 | 10 | 10 | 10 | 10 | 5 | 5 | 5 | 5 |

#1 Octyl gallate at 1 mg per ml was solubilized in 1 mM of phosphate baffer (pH 6.59) containing J1816 at 0.356 mg per ml.
#2 Propyl gallate at 10 mg per ml was solubilized in 1 mM of phosphate baffer (pH 6.59) containing J1816 at 0.356 mg per ml.

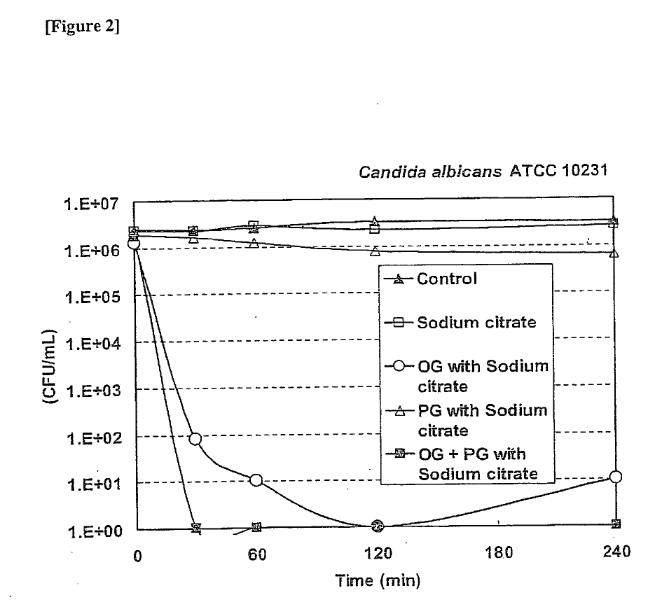

FIG. 2 shows shortening of the time required for killing mold by the concomitant use of octyl gallate and propyl gallate against Candida albicans ATCC 10231. It is found that the fungicidal activity of propyl gallate alone is low, however, by the concomitant use with octyl gallate, the time required for killing mold is shortened to a large extent.

Table 6 shows the reinforcement effect of isoamyl gallate or propyl gallate in the presence of 3% NaCl on the antimicrobial action of lauryl gallate against Pseudomonas aeruginosa POA1.

It is found that the antimicrobial action of lauryl gallate against Pseudomonas aeruginosa POA1 is reinforced by isoamyl gallate or propyl gallate in the presence of 3% NaCl.

TABLE 6

| Date | Feb. 2, 2007 | | | | | | | | | |
|---|---|---|---|---|---|---|---|---|---|---|
| MIC sample | Lauryl gallate MIC (μg/mL) | | | | | | | | | |
| medium | MHA (3% NaCl) | | | | | | | | | |
| concomitant | none | | Isoamyl gallate | | | | Propyl gallate | | | |
| conc. (μg/mL) | none | | 75 | | 50 | | 100 | | 75 | |
| time | 24 h | 48 h | 24 h | 48 h | 24 h | 48 h | 24 h | 48 h | 24 h | 48 h |
| P. aoruginosa PAO1 | 15.625 | 31.25 | ND | 0.9766 | ND | 3.9063 | ND | ND | ND | 0.4883 |

Table 7 shows the reinforcement effect of isoamyl gallate on the antimicrobial action of octyl gallate against clinical isolates MRSA and MSSA.

Table 7 shows that the antimicrobial action of octyl gallate against clinical isolates MRSA and MSSA is reinforced by isoamyl gallate. Octyl gallate and isoamyl gallate were solubilized with J1816 in an amount 3.5 times (weight ratio) larger than that for alkyl gallate.

TABLE 7

| Date | Mar. 8, 2007 | | | | | |
|---|---|---|---|---|---|---|
| MIC sample | Octyl gallate MIC (μg/mL) | | | | | |
| medium | CAMHA | | | | | |
| concomitant | J-1816 | | Isoamyl gallate | | | |
| conc. (μg/mL) | 262.5 | | 50 | | 25 | |
| time | 24 h | 48 h | 24 h | 48 h | 24 h | 48 h |
| MRSA #1 | 25 | 50 | ND | 12.5 | 12.5 | 25 |
| MRSA #2 | ND | ND | ND | ND | 0.3906 | 12.5 |
| MRSA #3 | ND | ND | ND | ND | ≦0.0244 | ≦0.0244 |
| MRSA #4 | 25 | 25 | ND | ND | 6.25 | 12.5 |
| MRSA #5 | ND | 6.25 | ND | ND | 3.125 | 3.125 |
| MRSA #6 | ND | 1.5625 | ND | ND | 3.125 | 12.5 |
| MRSA #7 | ND | ND | ND | ND | ≦0.0244 | 6.25 |
| MRSA #8 | ND | 25 | ND | ND | 0.3906 | 6.25 |
| MRSA #9 | 25 | 25 | ND | ND | 1.5625 | 12.5 |
| MRSA #10 | 0.3906 | 6.25 | ND | ND | 3.125 | 12.5 |
| MRSA #12 | ND | ND | ≦0.0244 | ≦0.0244 | 12.5 | 12.5 |
| MRSA #16 | 25 | 25 | ND | ND | 6.25 | 12.5 |
| MRSA #17 | 25 | 25 | ND | ND | 3.125 | 12.5 |
| MRSA #18 | 25 | 25 | ND | ND | 12.5 | 12.5 |
| MRSA #19 | 25 | 25 | ND | ND | 3.125 | 12.5 |
| MRSA #20 | ND | ND | ND | ND | 1.5625 | 6.25 |
| MRSA #21 | ND | ND | ND | ND | 12.5 | 12.5 |
| MRSA #22 | ND | ND | ND | ND | 12.5 | 25 |
| MRSA COL | ND | 0.7813 | ND | 0.1953 | 6.25 | 25 |
| MRSA #13 | ND | ND | ND | ND | 3.125 | 12.5 |
| MRSA Mu3 | 25 | 25 | ND | ND | 0.0488 | 12.5 |
| MSSA 1003 | ND | ND | ND | ND | 0.3906 | 3.125 |
| MSSA 1010 | ND | ND | ND | ND | 3.125 | 12.5 |
| MSSA 1020 | ND | ND | ND | ND | 3.125 | 6.25 |
| MSSA 1023 | ND | 25 | ND | ND | 1.5625 | 12.5 |
| MSSA 1029 | ND | 12.5 | ND | ND | 12.5 | 12.5 |
| MSSA 1032 | ND | ND | ND | ND | 12.5 | 12.5 |
| MSSA ATCC | ND | 50 | ND | 1.56 | 12.5 | 25 |
| MSSA RN | ND | ND | ND | ND | 0.195 | 12.5 |

### 2. Additional Reinforcement of the Enhancing Effect of β-Lactam Sensitivity to MRSA by Alkyl Gallates

It has been found that the alkyl gallates enhance the sensitivity of β-lactams to MRSA (PCT/JP2004/000751); further, it has been found that this enhancing effect can further be reinforced in the coexistence of an alkyl gallate in which the carbon number of the alkyl chain is smaller than that of octyl gallate. Table 8 and Table 9 show examples of propyl gallate and isoamyl gallate at a concentration at which no antimicrobial activity is observed, but in the coexistence of these gallates, a decrease of the MIC value of oxacillin to MRSA occurs by octyl gallate at a concentration as low as 1.56 μg/ml.

TABLE 8

| | Sep. 10, 2006 | | | | | | | | | | |
|---|---|---|---|---|---|---|---|---|---|---|---|
| | Oxacillin MIC (μg/mL) | | | | | | | | | | |
| | Isoamyl G or Propyl G | | | | | Isoamyl G (μg/mL) | | | | | |
| | none | | | | | 25 | | | 12.5 | | |
| | Octyl G (μg/mL) | | | | | Octyl G (μg/mL) | | | Octyl G (μg/mL) | | |
| | none | 12.5 | 6.25 | 3.125 | 1.5625 | 3.125 | 1.5625 | none | 3.125 | 1.5625 | none |
| 24 h | 24 h | 24 h | 24 h | 24 h | 24 h | 24 h | 24 h | 24 h | 24 h | 24 h | 24 h |
| MRSA #8 | 128 | 4 | 16 | 32 | 64 | ≦0.063 | 0.125 | 0.125 | 32 | 8 | 32 |
| MRSA #10 | 64 | 1 | 2 | 8 | 32 | 0.125 | 0.125 | 0.5 | 4 | 2 | 8 |
| MRSA COL | 256 | 1 | 16 | 64 | 64 | 0.125 | 0.125 | 0.25 | 32 | 4 | 8 |
| MRSA Mu3 | 512 | 32 | 64 | 256 | 512 | 0.25 | 0.25 | 0.5 | 64 | 64 | 256 |

| | Sep. 10, 2006 | | | | | | |
|---|---|---|---|---|---|---|---|
| | Oxacillin MIC (μg/mL) | | | | | | |
| | Isoamyl G (μg/mL) | | | | | Propyl G (μg/mL) | |
| | 6.25 | | | | | 25 | |
| | Octyl G (μg/mL) | | | | | Octyl G (μg/mL) | |
| | 12.5 | 6.25 | 3.125 | 1.5625 | none | 1.5625 | none |
| 24 h | 24 h | 24 h | 24 h | 24 h | 24 h | 24 h | 24 h |
| MRSA #8 | 16 | 32 | 16 | 128 | 64 | 0.125 | 1 |
| MRSA #10 | 1 | 2 | 16 | 32 | 32 | 0.25 | 1 |
| MRSA COL | 4 | 16 | 8 | 128 | 128 | 0.25 | 0.5 |
| MRSA Mu3 | 64 | 128 | 64 | 128 | 256 | 1 | 4 |

TABLE 9

| | Sep. 10, 2006 | | | | | | | | | | | | |
|---|---|---|---|---|---|---|---|---|---|---|---|---|---|
| | Oxacillin MIC (μg/mL) | | | | | | | | | | | | |
| | Isoamyl G or Propyl G | | | | | Isoamyl G | | | | | | | |
| | none | | | | | 25 | | | 12.5 | | | 6.25 | |
| | Octyl G | | | | | Octyl G | | | Octyl G | | | Octyl G | |
| | none | 12.5 | 6.25 | 3.125 | 1.5825 | 3.125 | 1.5825 | none | 3.125 | 1.5825 | none | 12.5 | 6.25 |
| 48 h | 48 h | 48 h | 48 h | 48 h | 48 h | 48 h | 48 h | 48 h | 48 h | 48 h | 48 h | 48 h | 48 h |
| MRSA #1 | 512 | 64 | 128 | 128 | 256 | 8 | 8 | 16 | 128 | 128 | 256 | 256 | 64 |
| MRSA #2 | 256 | 16 | 32 | 32 | 64 | 1 | 2 | 4 | 64 | 32 | 128 | 64 | 64 |
| MRSA #3 | 128 | 2 | 8 | 16 | 32 | 0.5 | 0.5 | 0.25 | 32 | 16 | 32 | 64 | 64 |
| MRSA #4 | 256 | 32 | 64 | 64 | 128 | 16 | 8 | 8 | 64 | 64 | 256 | 256 | 128 |
| MRSA #5 | 256 | 32 | 64 | 64 | 128 | 0.5 | 1 | 1 | 32 | 32 | 64 | 64 | 64 |
| MRSA #6 | 128 | 16 | 32 | 32 | 64 | 0.5 | 1 | 1 | 32 | 32 | 32 | 64 | 32 |
| MRSA #7 | 256 | 32 | 128 | 128 | 256 | 1 | 0.5 | 1 | 128 | 32 | 128 | 128 | 128 |
| MRSA #8 | 128 | 8 | 32 | 64 | 64 | 0.125 | 0.125 | 0.25 | 64 | 8 | 64 | 64 | 64 |
| MRSA #9 | 256 | 128 | 128 | 128 | 256 | 4 | 2 | 8 | 128 | 128 | 256 | 256 | 256 |
| MRSA #10 | 128 | 8 | 16 | 16 | 32 | 0.5 | 1 | 2 | 16 | 16 | 32 | 16 | 16 |
| MRSA #12 | 256 | 16 | 32 | 64 | 64 | 2 | 2 | 2 | 32 | 32 | 64 | 32 | 32 |
| MRSA #16 | 256 | 32 | 64 | 64 | 64 | 0.5 | 0.5 | 1 | 32 | 16 | 64 | 32 | 32 |
| MRSA #17 | 128 | 16 | 32 | 32 | 84 | 1 | 2 | 2 | 32 | 16 | 32 | 64 | 32 |
| MRSA #18 | 256 | 32 | 64 | 128 | 128 | 16 | 8 | 32 | 64 | 64 | 128 | 256 | 128 |
| MRSA #19 | ND | ND | ND | ND | ND | ND | ND | ND | ND | ND | ND | ND | ND |
| MRSA #20 | 64 | 4 | 8 | 4 | 16 | 0.125 | 0.25 | 0.5 | 8 | 2 | 2 | 8 | 8 |
| MRSA #21 | 64 | 0.5 | 4 | 18 | 32 | 0.5 | 0.5 | 1 | 16 | 8 | 16 | 16 | 16 |
| MRSA #22 | 64 | 2 | 8 | 8 | 16 | 0.25 | 0.25 | 1 | 16 | 4 | 8 | 16 | 16 |
| MRSA COL | 256 | 4 | 32 | 64 | 64 | 0.25 | 0.25 | 1 | 32 | 4 | 32 | 16 | 32 |
| MSSA 1003 | 0.5 | 0.25 | 0.5 | 0.5 | 0.5 | 0.125 | 0.125 | 0.25 | 0.5 | 0.5 | 0.5 | 0.5 | 0.5 |
| MSSA 1010 | 0.5 | 0.125 | 0.25 | 0.5 | 0.5 | 0.125 | 0.125 | 0.25 | 0.25 | 0.5 | 0.5 | 0.25 | 0.25 |
| MSSA 1020 | 2 | 0.6 | 1 | 2 | 2 | 0.25 | 0.25 | 0.5 | 1 | 1 | 2 | 0.6 | 1 |
| MSSA 1023 | 8 | 4 | 4 | 4 | 8 | 0.5 | 0.5 | 1 | 8 | 2 | 4 | 4 | 4 |
| MSSA 1029 | 2 | 0.5 | 1 | 2 | 2 | 0.25 | 0.25 | 0.5 | 1 | 1 | 1 | 0.5 | 1 |
| MSSA 1032 | 0.25 | 0.125 | 0.25 | 0.25 | 0.25 | 0.125 | 0.25 | 0.25 | 0.25 | 0.25 | 0.25 | 0.125 | 0.25 |
| MSSA ATCC | 0.125 | 30.003 | 0.125 | 0.125 | 0.125 | 0.25 | 0.25 | 0.25 | 0.25 | 0.25 | 0.25 | 0.25 | 0.25 |
| MSSA RN | 0.5 | 0.25 | 0.5 | 0.5 | 0.5 | 0.125 | 0.125 | 0.25 | 0.5 | 0.5 | 0.5 | 0.125 | 0.25 |
| MRSA #13 | 256 | 16 | 128 | 128 | 256 | 1 | 1 | 2 | 128 | 64 | 128 | 128 | 128 |
| MRSA Mu3 | 512 | 64 | 256 | 256 | 512 | 1 | 0.25 | 8 | 128 | 64 | 256 | 256 | 256 |

| | Sep. 10, 2006 | | | | | | Sep. 10, 2006 | | | | | |
|---|---|---|---|---|---|---|---|---|---|---|---|---|
| | Oxacillin MIC (μg/mL) | | | | | | Octyl gallate MIC (μg/mL) | | | | | |
| | Isoamyl G | | | Propyl G | | | | | | | | |
| | 6.25 | | | 25 | | | | | | | | |
| | Octyl G | | | Octyl G | | | Isoamyl G | | | | | |
| | 3.125 | 1.5625 | none | 1.5625 | none | | 25 | | | 12.5 | 6.25 | none |
| | | | | | | | | Route of | | | | |
| | | | | | | | | inhibition | | | | |
| 48 h | 48 h | 48 h | 48 h | 48 h | 48 h | 48 h | 48 h | concentration | | 48 h | 48 h | 48 h |
| MRSA #1 | 256 | 256 | 256 | 32 | 32 | MRSA #1 | 3.906 | | | 31.25 | 31.25 | 31.25 |
| MRSA #2 | 128 | 128 | 128 | 8 | 2 | MRSA #2 | 0.4883 | | | 31.25 | 31.25 | 31.25 |
| MRSA #3 | 64 | 64 | 64 | 1 | 1 | MRSA #3 | 0.9756 | | | 31.25 | 31.25 | 62.5 |
| MRSA #4 | 128 | 128 | 128 | 8 | 8 | MRSA #4 | 15.625 | | | 31.25 | 31.25 | 31.25 |
| MRSA #5 | 256 | 128 | 128 | 4 | 4 | MRSA #5 | 0.4883 | | | 31.25 | 31.25 | 31.25 |
| MRSA #6 | 64 | 128 | 64 | 1 | 2 | MRSA #6 | 1.953 | | | 31.25 | 31.25 | 31.25 |
| MRSA #7 | 128 | 256 | 256 | 4 | 16 | MRSA #7 | 31.25 | 3.906 | | 31.25 | 31.25 | 31.25 |
| MRSA #8 | 32 | 128 | 128 | 1 | 4 | MRSA #8 | 0.2441 | | | 31.25 | 31.25 | 31.25 |
| MRSA #9 | 256 | 258 | 256 | 4 | 4 | MRSA #9 | 0.0758 | | | 31.25 | 31.25 | 31.25 |
| MRSA #10 | 32 | 64 | 64 | 4 | 4 | MRSA #10 | ND | | | 7.8125 | 31.25 | 31.25 |
| MRSA #12 | 64 | 128 | 128 | 4 | 4 | MRSA #12 | 31.25 | 3.906 | 7.8125 | 31.25 | 31.25 | 31.25 |
| MRSA #16 | 32 | 128 | 128 | 2 | 4 | MRSA #16 | 0.4883 | | | 31.25 | 31.25 | 31.25 |
| MRSA #17 | 32 | 64 | 64 | 4 | 4 | MRSA #17 | 7.8125 | | | 31.25 | 31.25 | 31.25 |
| MRSA #18 | 128 | 256 | 128 | 8 | 8 | MRSA #18 | 15.526 | | | 31.25 | 31.25 | 31.25 |
| MRSA #19 | ND | ND | ND | ND | ND | MRSA #19 | ND | | | ND | ND | ND |
| MRSA #20 | 16 | 32 | 32 | 0.5 | 1 | MRSA #20 | 0.2441 | | | 3.906 | 31.25 | 31.25 |
| MRSA #21 | 32 | 64 | 32 | 12 | 0.5 | MRSA #21 | 31.25 | | | 31.25 | 31.25 | 31.25 |
| MRSA #22 | 16 | 32 | 32 | 0.5 | 0.5 | MRSA #22 | 0.2441 | | | 31.25 | 31.25 | 31.25 |
| MRSA COL | 32 | 128 | 128 | 1 | 1 | MRSA COL | 0.4883 | | | 31.25 | 31.25 | 31.25 |
| MSSA 1003 | 0.5 | 0.5 | 0.5 | 0.25 | 0.125 | MSSA 1003 | 0.1221 | | | 3.906 | 31.25 | 31.25 |
| MSSA 1010 | 0.5 | 0.5 | 0.5 | 0.5 | 0.5 | MSSA 1010 | 50.0810 | | | 0.0766 | 7.8125 | 31.25 |
| MSSA 1020 | 2 | 2 | 2 | 0.5 | 0.5 | MSSA 1020 | 31.25 | 3.906 | 7.8125 | 31.25 | 31.25 | 31.25 |
| MSSA 1023 | 4 | 8 | 8 | 1 | 1 | MSSA 1023 | 0.2441 | | | 31.25 | 31.25 | 31.25 |
| MSSA 1029 | 1 | 2 | 1 | 0.5 | 1 | MSSA 1029 | ND | | | 31.25 | 31.25 | 31.25 |
| MSSA 1032 | 0.25 | 0.25 | 0.25 | 0.125 | 0.25 | MSSA 1032 | 0.4883 | | | 31.25 | 31.25 | 31.25 |
| MSSA ATCC | 0.25 | 0.25 | 0.125 | 0.25 | 0.25 | MSSA ATCC | 31.25 | | | 31.25 | 31.25 | 31.25 |
| MSSA RN | 0.5 | 0.5 | 0.5 | 0.25 | 0.5 | MSSA RN | 50.0010 | | | 31.25 | 31.25 | 31.25 |
| MRSA #13 | 128 | 128 | 128 | 8 | 8 | MRSA #13 | 31.25 | 3.906 | 7.8125 | 31.25 | 31.25 | 31.25 |
| MRSA Mu3 | 128 | 128 | 256 | 16 | 128 | MRSA Mu3 | 3.906 | | | 31.25 | 31.25 | 31.25 |

This time, in several cases, the growth of bacterium was once inhibited, then began again at a higher concentration, and then inhibited again.
MIC was determined at a concentration at which the bacterium did not grow again.
When the inhibition of growth was observed at a lower concentration than MIC, the growth inhibition concentration at the lowest concentration was indicated in the left column of “Range of inhibition concentration”, and the inhibition concentration immediately before the growth of bacterium began again was indicated in the right column.

Table 10 shows the MIC value of oxacillin when it was used alone and the MIC value of oxacillin when it was used concomitantly with octyl gallate for the clinical isolate MRSA.

To be more specific, the MIC value of oxacillin when it was used alone and the MIC value of oxacillin when it was used concomitantly with octyl gallate determined by the 2-fold serial agar plate dilution method are shown for the respective bacterial strains tested. An ILSMR effect could be confirmed in several bacterial strains when the concomitant use of octyl gallate at 12.5 μg/mL, and a growth inhibitory effect was observed in all the bacterial strains with only octyl gallate when the concomitant use of octyl gallate at 25 μg/mL.

TABLE 10
Oxacillin MIC against clinical isolate MRSA when
oxacillin was used alone and when oxacillin was used
concomitantly with octyl gallate

Oxacillin MIC (μg/mL)
Octyl gallate (μg/mL)

| Strain | None | 25 | 12.5 | 6.25 |
|---|---|---|---|---|
| MRSA #1 | 512 | ND | 128 | 256 |
| MRSA #2 | 256 | ND | 128 | 128 |
| MRSA #3 | 128 | ND | 64 | 128 |
| MRSA #4 | 256 | ND | 128 | 256 |
| MRSA #5 | 256 | ND | 128 | 256 |
| MRSA #6 | 128 | ND | 32 | 64 |
| MRSA #7 | 256 | ND | 256 | 256 |
| MRSA #8 | 256 | ND | 64 | 128 |
| MRSA #9 | 256 | ND | 128 | 256 |
| MRSA #10 | 128 | ND | 64 | 128 |
| MRSA #12 | 256 | ND | 128 | 256 |
| MRSA #13 | 256 | ND | 128 | 256 |
| MRSA #16 | 256 | ND | 128 | 256 |
| MRSA #17 | 256 | ND | 64 | 128 |
| MRSA #18 | 256 | ND | 256 | 256 |
| MRSA #19 | 256 | ND | 128 | 256 |
| MRSA #20 | 32 | ND | 4 | 32 |
| MRSA #21 | 64 | ND | 16 | 64 |
| MRSA #22 | 64 | ND | 16 | 32 |
| MRSA COL | 512 | ND | 128 | 256 |
| MRSA Mu3 | 512 | ND | 256 | 512 |

Table 11 shows the concentrations of octyl gallate necessary for obtaining an oxacillin MIC value not higher than 2 μg/mL when oxacillin was used concomitantly with octyl gallate for the respective bacterial strains. To be more specific, the table shows the case where oxacillin was used concomitantly only with octyl gallate without the coexistence of a short-chain gallate and the case where oxacillin was used concomitantly with isoamyl gallate or propyl gallate in addition to octyl gallate. It is found that when 25 μg/mL of isoamyl gallate is allowed to coexist, the concomitant use of octyl gallate at 1.56 μg/mL is sufficient in order to obtain oxacillin MIC of 2 μg/mL.

TABLE 11
Reinforcement effect of concomitant use of octyl gallate in the presence of
short-chain alkyl gallate on oxacillin sensitivity to clinical isolate MRSA

Concentration of octyl gallate for concomitant use
necessary for obtaining oxacillin MIC not higher
than 2 μg/mL (μg/mL)

| | |
|---|---|
| Isoamyl gallate (μg/mL) | Propyl gallate (μg/mL) |

| Strain | None | 25 | 12.5 | 6.25 | 25 | 12.5 | 6.25 |
|---|---|---|---|---|---|---|---|
| MRSA #1 | 25 | 1.56 | >12.5 | >12.5 | 6.25 | >12.5 | >12.5 |
| MRSA #2 | 25 | 1.56 | 12.5 | >12.5 | 3.13 | >12.5 | >12.5 |
| MRSA #3 | 25 | 1.56 | >12.5 | >12.5 | 3.13 | >12.5 | >12.5 |
| MRSA #4 | 25 | 1.56 | >12.5 | >12.5 | ND | ND | ND |
| MRSA #5 | 25 | 1.56 | >12.5 | >12.5 | 6.25 | >12.5 | >12.5 |
| MRSA #6 | 25 | 1.56 | >12.5 | >12.5 | 6.25 | >12.5 | >12.5 |
| MRSA #7 | 25 | 1.56 | 12.5 | >12.5 | 3.13 | >12.5 | >12.5 |
| MRSA #8 | 25 | 1.56 | 6.25 | >12.5 | 3.13 | >12.5 | >12.5 |
| MRSA #9 | 25 | 1.56 | 12.5 | >12.5 | 3.13 | >12.5 | >12.5 |
| MRSA #10 | 25 | 1.56 | 1.56 | 6.25 | 3.13 | >12.5 | >12.5 |
| MRSA #12 | 25 | 1.56 | >12.5 | >12.5 | 6.25 | >12.5 | >12.5 |
| MRSA #13 | 25 | 1.56 | >12.5 | >12.5 | 6.25 | >12.5 | >12.5 |
| MRSA #16 | 25 | 1.56 | >12.5 | >12.5 | 6.25 | >12.5 | >12.5 |
| MRSA #17 | 25 | 1.56 | 12.5 | >12.5 | 6.25 | >12.5 | >12.5 |
| MRSA #18 | 25 | 1.56 | >12.5 | >12.5 | ND | ND | ND |
| MRSA #19 | 25 | 1.56 | >12.5 | >12.5 | 6.25 | >12.5 | >12.5 |
| MRSA #20 | 25 | 1.56 | 1.56 | >12.5 | 3.13 | >12.5 | >12.5 |
| MRSA #21 | 25 | 1.56 | 6.25 | >12.5 | 1.56 | 6.25 | 12.5 |
| MRSA #22 | 25 | 1.56 | 6.25 | >12.5 | 1.56 | 6.25 | 12.5 |
| MRSA COL | 25 | 1.56 | >12.5 | >12.5 | 1.56 | 12.5 | 12.5 |
| MRSA Mu3 | 25 | 1.56 | >12.5 | >12.5 | 6.25 | >12.5 | >12.5 |

Table 12 shows an effect of concomitant use of a short-chain alkyl gallate on reinforcement effect of octyl gallate on oxacillin sensitivity to the clinical isolate MRSA, and the concentrations of octyl gallate necessary for obtaining an oxacillin MIC value not higher than 2 μg/mL shown in Table 7 are summarized in Table 12 in terms of Range, C₅₀ and C₁₀₀. C₅₀ and C₁₀₀ denote the concentrations of octyl gallate for concomitant use, which achieve 50% and 100% growth inhibition of bacterial strains by oxacillin at 2 μg/mL or lower, respectively. When propyl gallate was allowed to coexist at 25 μg/mL, 100% growth inhibition of MRSA strain was achieved by oxacillin at 2 μg/mL or lower with the concomitant use of octyl gallate at 6.25 μg/mL. When isoamyl gallate was allowed to coexist at 25 μg/mL, 100% growth inhibition of MRSA strain was achieved by oxacillin at 2 μg/mL or lower with the concomitant use of octyl gallate at 1.56 μg/mL. In this connection, an oxacillin MIC value of 2 μg/mL is used as an index for the sensitive strain (MSSA).

TABLE 12
Effect of concomitant use of short-chain alkyl gallate
on activity of reinforcing oxacillin sensitivity to clinical isolate
MRSA by octyl gallate

| | | Concentration of octyl | | |
|---|---|---|---|---|
| | | gallate for concomitant | | |
| | | use necessary for | | |
| | | obtaining oxacillin MIC | | |
| | | not higher than 2 | | |
| | | μg/mL (μg/mL) | | |
| | Conc. | Range | C₅₀ | C₁₀₀ |
| None | | 25 | 25 | 25 |
| Propyl | 6.25 | 12.5->12.5 | >12.5 | >12.5 |
| gallate | 12.5 | 6.25-12.5 | >12.5 | >12.5 |
| | 25 | 1.56-6.25 | 3.13 | 6.25 |
| Isoamyl | 6.25 | 6.25->12.5 | >12.5 | >12.5 |
| gallate | 12.5 | 1.56->12.5 | >12.5 | >12.5 |
| | 25 | 1.56 | 1.56 | 1.56 |

### 3. Reinforcement of Virucidal Activity and Anti-Viral Activity

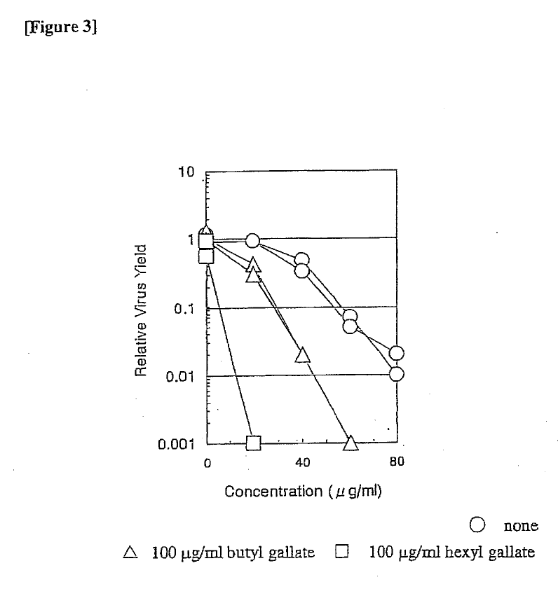

### 1) Reinforcement of Virucidal Activity (FIG. 3)

The open circle in FIG. 3 indicates an influenza virucidal effect of dodecyl gallate alone. The effect in the coexistence of 100 μg/ml of hexyl gallate is indicated by the open square in FIG. 3; thus, the virucidal effect of dodecyl gallate was markedly reinforced and no survivor was observed even at 20 μg/ml. This virucidal effect of dodecyl gallate was also reinforced markedly by the addition of butyl gallate (open triangle) at a concentration at which no virucidal effect was observed. As a result of elucidation in diverse ways, it was revealed that the virucidal activity of alkyl gallate A (in which the carbon number of the alkyl chain was 5 to 16) was reinforced by alkyl gallate B in which the carbon number of the alkyl chain was smaller than that of alkyl gallate A.

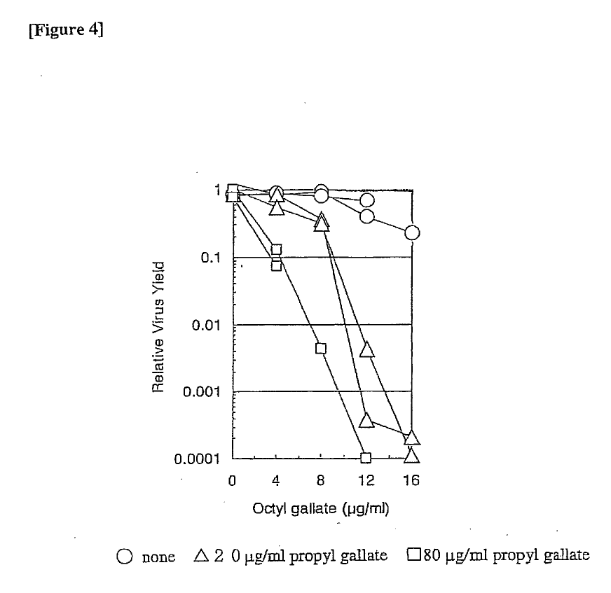

### 2) Reinforcement of Anti-Viral Activity (FIG. 4)

Octyl gallate inhibits the growth of influenza virus in MDCK cells; this growth inhibition was markedly reinforced by propyl gallate at a concentration at which no anti-viral activity was observed.

As a result of elucidation in diverse ways, it was revealed that the anti-viral activity of alkyl gallate A (in which the carbon number of the alkyl chain was 5 to 16) was reinforced by alkyl gallate B in which the carbon number of the alkyl chain was smaller than that of alkyl gallate A.

Table 13 indicates the target bacteria, fungi and viruses of the invention.

TABLE 13

Gram-positive bacteria
Actinomyces israelii
Bacillus anthracis
Bacillus cereus
Clostridium batulinum
Clostridium difficile
Clostridium perfringens
Clostridium tatani
Corynebacterium diphtherise
Enterococci
Gardnerella vaginalis
Listeria monocytogenes
Stephylococcus aureus (coagulase positive)
Stephylococcus spidermidis (coagulase negative)
Streptococcus agalactiae
Streptococcus mutans
Streptococcus pneumoniae
Streptococcus pyogenes
Mycobacterium tuberculosis
Mycobacterium leprae
Mycobacteria other than tuberculosis (M.O.T.T.)
Nocardio asteroides
Prepionibacterium acnes
Propionibacterium granulosus
Gram-negative bacteria
Bacteroides fragilis
Bartonella henselao
Bordetella pertussis
Borrelia burgoorferi
Borrelia recurrentis
Brucella suis
Burkholderia cepacia
Burkholderia pseudomallei
Campylobacter jejuni
Chlemydia pneumoniae
Chlamydia trachomatis
Escherichia coli
Francisella tularensis
Haemophilus ducreyi
Haemophilus influenzae
Helicobacter pylori
Legionella pneumophila
Morexella catarrhalis
Mycoplasma pneumoniae
Neisseria gonorrhoeao
Neisseria meningitidis
Pasteurella multocida
Pseudomonas aeruginosa
Riokettsia prowazekii
Riokettsia rickettsii
Salmonella enteritidis
Salmonella typhi
Shigella sonnai
Treponama pallidum
Ureaplasma urealyticum
Vibrio cholerae
Vibrio parahaemolyticus
Yersinia enterocolibico
Yersinia pestis
Yersinia pseudotuberculosis
Porphyromonas gingivalis
Viruses
Bovine Spongiform Encephalopathy(BSE)
Creutzfeldt-Jakob Disease(CJD)
Cytomegalovirus(CMV)
Corona virus
Dengue Virus
Ebola Virus
Enteroviruses(poliovirus)
Epstain-Barr Virus
Hentavirus
Hepatitis A Virus(HAV)
Hepatitis B Virus(HBV)
Hepatitis C Virus(HCV)
Herpes Simplex Virus(HSV) 1 & 2
Human Immunodeficiency Virus(HIV)
Human Papillomavirus(HPV)
Influenza Virus
Measles Virus
Mumps Virus
Nipah Virus
Noro virus
Rabies Virus
Respiratory Syncytial Virus(RSV)
Rhinovirus
Rubella Virus
SARS Corona virus
Varioella-Zoster Virus
West Nile Virus
Yellow Fever Virus
Fungi
Candida albicans
Coccidicides immitis
Cryptococcus neoformans
Dermatophytes (Trichophyton, Microsporium, Epidermophyton)
Histoplasma capsulatum
Pneumocystis carinii
Sporothrix schenckii

Tables 14 to 16 show that the virucidal activity of octyl gallate against herpes virus (HSV-1) and influenza virus was markedly reinforced by the addition of J1816 and propyl gallate.

In the coexistence of 6 mg/L of J1816, HSV-1 completely lost the infectivity to cells due to octyl gallate at 2 mg/L (20 mg/L in the absence of J1816). Similarly, also in the case of influenza virus, in the presence of 30 mg/L of J1816, the influenza virus completely lost the infectivity to cells due to octyl gallate at 10 mg/L (60 mg/L in the absence of J1816). Accordingly, it is found that the virucidal activity of octyl gallate against herpes virus (HSV-1) and influenza virus was markedly reinforced by the addition of J1816.

TABLE 14
Virucidal activity against HSV-1 by the coexistence of octyl
gallate, J1816 and propyl gallate

| #1octyl | #2propyl | J1816 | | |
|---|---|---|---|---|
| gallate (mg/L) | gallate (mg/L) | (mg/L) | Na3citrate | No of plaques |
| 0 | | 0 | 0.90% | 182 |
| 10 | | 30 | 0.90% | 0 |
| 20 | | 60 | 0.90% | 0 |
| 30 | | 90 | 0.90% | 0 |
| 60 | | 180 | 0.90% | 0 |
| 100 | | 300 | 0.90% | 0 |
| 0 | 300 | 90 | 0.90% | 0 |
| 10 | 300 | 120 | 0.90% | 0 |
| 20 | 300 | 150 | 0.90% | 0 |
| 30 | 300 | 180 | 0.90% | 0 |
| 60 | 300 | 270 | 0.90% | 0 |
| 100 | 300 | 390 | 0.90% | 0 |
| | 0 | | 0.90% | 128 |
| | 100 | 30 | 0.90% | 0 |
| | 200 | 60 | 0.90% | 0 |
| | 300 | 90 | 0.90% | 0 |
| | 400 | 120 | 0.90% | 0 |
| | 500 | 150 | 0.90% | 0 |
| 60 | | 180 | | 0 |
| | | 150 | 0.90% | 0 |
| | | 300 | 0.90% | 0 |

#1Octyl gallate (1 mg/ml) was solbilized in 1 mM phosphate buffer containing 3 mg/ml of J1816.
#2Propyl gallate (5 mg/ml) was solubilized in 5 mM phosphate buffer containing 1.5 mg/ml of J1816

TABLE 15
Virucidal activity against HSV-1 by the coexistence of octyl
gallate and propyl gallate

| #3octyl | propyl gallate | J1816 | | |
|---|---|---|---|---|
| gallate (mg/L) | (mg/L [symbol] | (mg/L) | NaCitrate | No of plaques |
| 0 | | 0 | 0.90% | 158 |
| 10 | | 0 | 0.90% | 48 |
| 20 | | 0 | 0.90% | 0 |
| 30 | | 0 | 0.90% | 0 |
| 60 | | 0 | 0.90% | 0 |
| 100 | | 0 | 0.90% | 0 |
| 0 | 300 | 0 | 0.90% | 137 |
| 10 | 300 | 0 | 0.90% | 11 |
| 20 | 300 | 0 | 0.90% | 0 |
| 30 | 300 | 0 | 0.90% | 0 |
| 60 | 300 | 0 | 0.90% | 0 |
| 100 | 300 | 0 | 0.90% | 0 |
| | 0 | 0 | 0.90% | 150 |
| | 100 | 0 | 0.90% | 192 |
| | 200 | 0 | 0.90% | 139 |
| | 300 | 0 | 0.90% | 162 |
| | 400 | 0 | 0.90% | 102 |
| | 500 | 0 | 0.90% | 130 |

#3Octyl gallate was solubilized with 1M Arginine
#4: Propyl gallate (3 mg/ml) was solubilized in 5 mM phosphate buffer at pH 6.71 [symbol]
indicates data missing or illegible when filed

TABLE 16
Virucidal activity against influenza virus A/Aichi (H3N2)
by the coexistence of octyl gallate, J1816 and propyl gallate

| | #1 octyl ga | #2propyl gallte (mg/L | J1816 (mg/L) | NaCitrate | No of plaques | | |
|---|---|---|---|---|---|---|---|
| 1 | 0 | | 0 | 0.90% | 256 | 244 | 248 |
| 2 | 20 | | 60 | 0.90% | 0 | | |
| 3 | 40 | | 20 | 0.90% | 0 | | |
| 4 | 60 | | 180 | 0.90% | 0 | | |
| 5 | 80 | | 240 | 0.90% | 0 | | |
| 6 | 100 | | 300 | 0.90% | 0 | | |
| 7 | 0 | 300 | 90 | 0.90% | 52 | | |
| 8 | 20 | 300 | 150 | 0.90% | 0 | | |
| 9 | 40 | 300 | 210 | 0.90% | 0 | | |
| 10 | 60 | 300 | 270 | 0.90% | 0 | | |
| 11 | 80 | 300 | 330 | 0.90% | 0 | | |
| 12 | 100 | 300 | 390 | 0.90% | 0 | | |
| 13 | | 0 | 0 | 0.90% | 214 | | |
| 14 | | 100 | 30 | 0.90% | 84 | | |
| 15 | | 200 | 60 | 0.90% | 87 | | |
| 16 | | 300 | 90 | 0.90% | 53 | | |
| 17 | | 400 | 120 | 0.90% | 47 | | |
| 18 | | 500 | 150 | 0.90% | 42 | | |
| 19 | 60 | | 180 | | 0 | | |
| 20 | | | 50 | | 118 | | |
| 21 | | | 100 | | 100 | | |
| 22 | | | 150 | | 84 | | |
| 23 | | | 200 | | 78 | | |
| 24 | | | 300 | | 88 | | |
| 25 | | | 400 | | — | | |
| 26 | | | 500 | | 76 | | |

#1: Octyl gallate (1 mg/ml) was solbilized in 1 mM phosphate buffer containing 3 mg/ml of J1816.
#2: Propyl gallate (5 mg/ml) was solubilized in 5 mM phosphate buffer containing 1.5 mg/ml of J1816

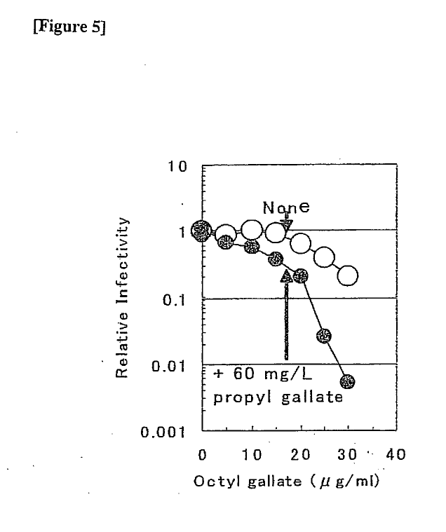

FIG. 5 shows that a marked reinforcement of virucidal activity of octyl gallate against HSV-1 was observed by 60 mg/L of propyl gallate.

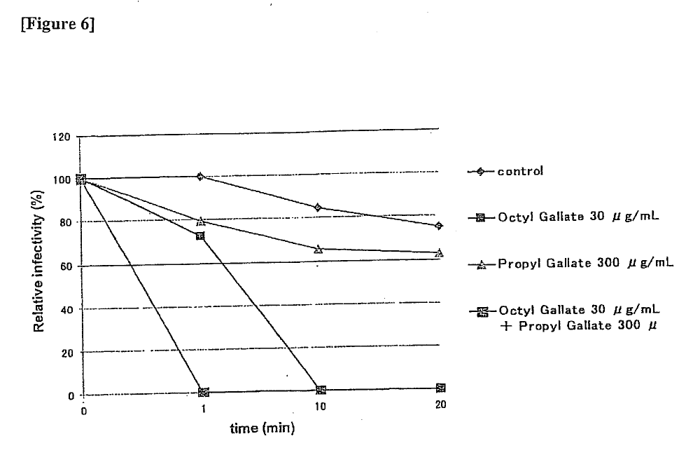

FIG. 6 shows shortening of the time required for killing viruses by the concomitant use of octyl gallate and propyl gallate against influenza B/T/1/05. It is found that the virucidal activity of propyl gallate alone is low, however, by the concomitant use of octyl gallate, the time required for killing viruses is shortened to a large extent.

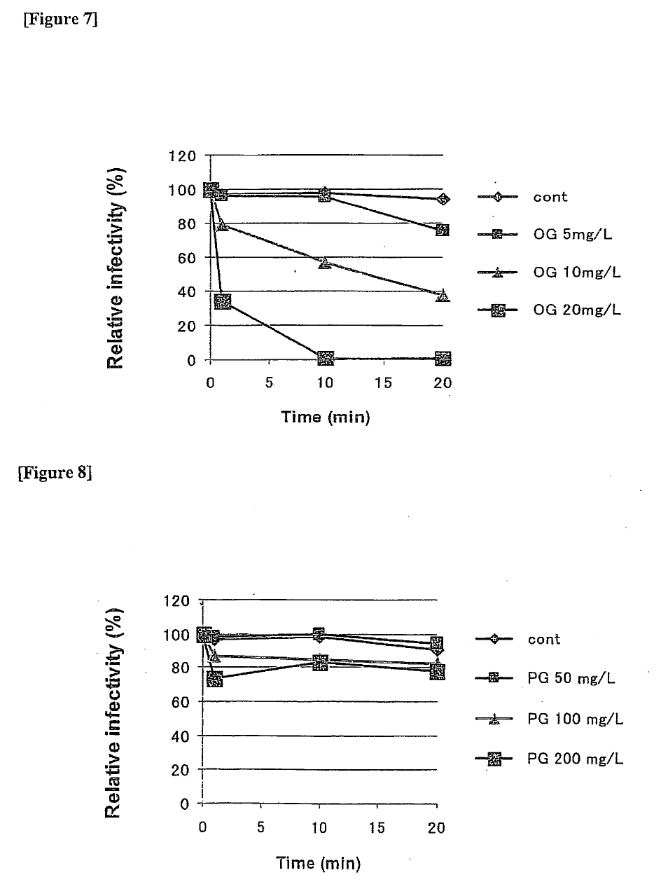

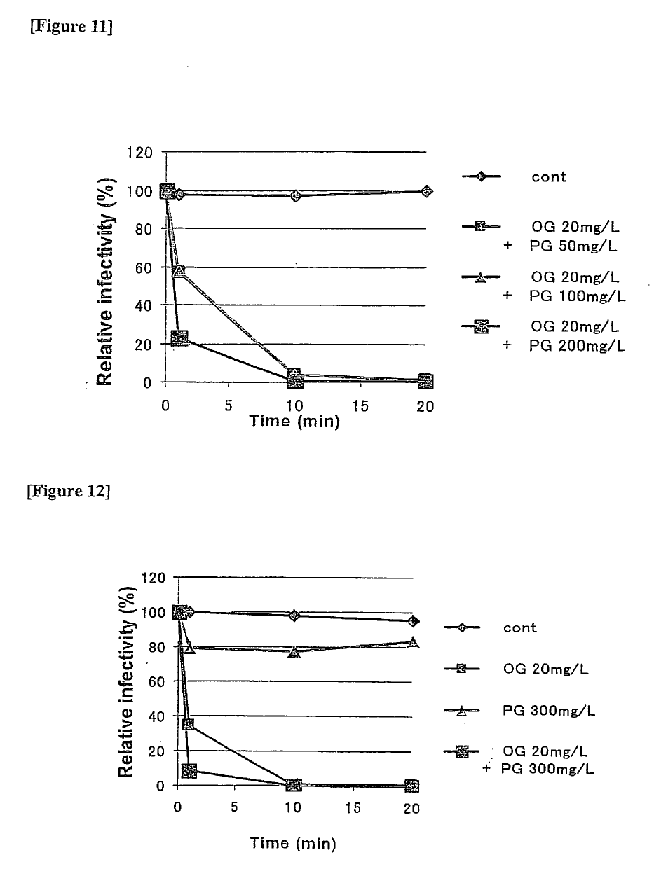

The experiments in FIGS. 7 to 12 were carried out at 37° C. The infectivity of viruses was determined using MDCK cells by the plaque assay method.

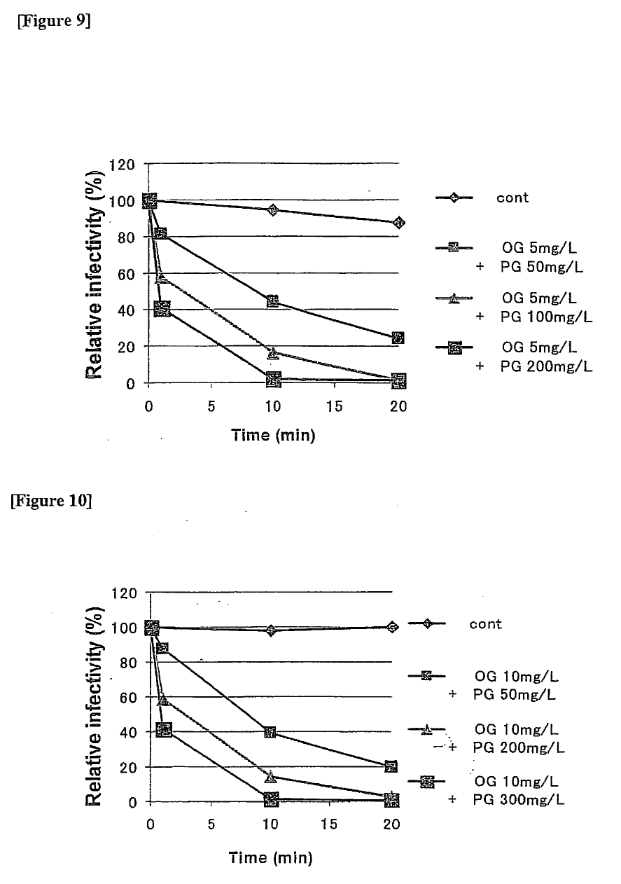

It is found that from FIG. 7, octyl gallate has a virucidal activity against influenza virus B/T/1/05, and from FIG. 8, propyl gallate has a low virucidal activity. However, FIGS. 9 to 12 show that the virucidal activity of octyl gallate is markedly reinforced by the concomitant use with propyl gallate. Further, in FIG. 12, it is found that by the concomitant use of both compounds, the infectivity to cells lost within 1 minute, therefore, it can be expected that an extremely potent virucidal agent is formed. This virucidal activity of octyl gallate was reinforced by propyl gallate also against influenza virus A/Aichi (H₃N₂) in the same manner as above.

### 4. Scope of Application

Having potent anti-fungal, anti-bacterial, anti-viral effects with low toxicity, a wide variety of the following applications relative to pharmaceuticals, agrochemicals (domestic animals, hatchery fishes, pets, plants), cosmetics and functional food products are possible.

1) General external sterilization and disinfection (disinfection of surgical instruments and medical instruments, disinfection of medical care facility, disinfection of hands), prevention of in-hospital infection
2) Otolaryngological field (eradication of intranasal MRSA, and the like)
3) Dermatological field (prevention and therapy of bed sore, thermal burn and acne, elimination of body odor), cosmetics for cure of acne, shampoo and body shampoo, and the like
4) Oral dental field (prevention and therapy of common cold, prevention and therapy of pharyngitis, therapy and prevention of dental caries, therapy and prevention of periodontitis, elimination and prevention of halitosis, therapy and prevention of stomatitis); gargle, medical tooth powder, mouth wash
5) Ophthalmic field (therapy and prevention of bacterial, fungal or viral infection, sterilization and disinfection of contact lens); eye drops, disinfectants
6) Gynecological field (anti-bacterial, anti-fungal, anti-viral sanitary goods, virucidal agents for HIV and the like)
7) Field of food poisoning (therapy and prevention of food poisoning caused by Vibrio parahaemolyticus, Campylobacter jejuni/coli, Salmonella, Echerichia coli, Clostridium perfringens, Bacillus cereusu, Yersinia enterocolitica, Vibrio cholerae, Vibrio mimicus, Vibrio fluvialis, Aeromonas hydrophila, Aeromonas sobria, Plesimonas shigelloides, Staphylococcus aureus, Clostridium botulinum, Norovirus, and the like); therapeutic and preventive agents for food poisoning
8) Therapeutic agents for pneumonia (therapy and prevention of pneumonia caused by Mycoplasma pneumoniae, Streptococcus pneumoniae, Hemophilus influennzae, Klebsiella pneumoniae, Legionella pneumophila, Moraxella catarrhalis, Staphylococcus aureus, Mycobacteria tuberculosis, a variety of viruses), therapy and prevention by inhalation
9) Functional food products (elimination of halitosis or prevention and therapy of common cold and stomatitis by adding to gum or the like)

Examples of the administration route of the pharmaceutical composition having an anti-fungal, anti-viral or anti-bacterial effect of the invention include parenteral administration, oral administration, local administration and the like in the same manner as in usual antibiotics. In general, administration by injection is preferred. In such a case, the injection may be prepared in a conventional manner, and a case where the ingredients are dissolved in a proper vehicle, for example, sterilized distilled water, physiological saline, or the like as a form of injection is also included.

Oral administration is also allowable in various dosage forms. For example, tablets, capsules, tablets coated with sugar or the like, and liquid solutions or suspensions are included in such forms.

The dose of the above-mentioned active ingredient used for prevention or therapy may be changed depending on the age, body weight, condition of the patient, and administration route; for example, the active ingredient may be administered orally at a dose of 1 mg to 3 g (per 1 kg of body weight) 1 to 3 times a day for adults. In order to obtain the best therapeutic effect, the dose and administration route are changed.

The pharmaceutical compositions of the invention are usually prepared according to a conventional method and formulated into a pharmaceutically suitable form. For example, a solid formulation may contain together with an active compound a diluent such as lactose, dextrose, saccharose, cellulose, corn starch or potato starch; a lubricant such as silica, talc, stearic acid, magnesium stearate or calcium stearate and/or polyethylene glycol; a biding agent such as starch, gum arabic, gelatin, methylcellulose, carboxymethylcellulose or polyvinylpyrrolidine; a disintegrator such as starch, alginic acid, an alginate or glycolic acid starch sodium; a foaming agent; a pigment; a sweetener; a wetting agent such as lecithin, polysorbate, or lauryl sulfate; and a generally non-toxic and pharmaceutically prescribed, pharmaceutically inactive substance. These pharmaceutical compositions may be produced according to a known process, for example, mixing, granulation, tablet formation, sugar coating, coating process or the like.

In parenteral administration, the most widely used formulation is of injection, though suppositories targeted for the rectum may also be employed. The preparations for injection include those different in external appearance, such as liquid preparations, preparations dissolving immediately before use, and suspension-type preparations, but basically they are considered to be the same because an active ingredient is sterilized in a proper way, then placed directly in a vessel, and tightly closed therein.

As one of the simplest methods for preparing a formulation, there is a method in which an active ingredient is sterilized in a proper way, then mixed separately or physically, and a certain amount of the resulting mixture is divided to yield a formulation. When a liquid form is chosen, a method in which an active ingredient is dissolved in a proper vehicle, sterilized by filtration, put into proper ampoules or vials, and tightly closed therein can be employed.

In this case, the most frequently used vehicle is distilled water for injection, but the invention is not restricted by it. If required, it is possible to add a soothing agent having a locally anesthetic action such as procaine hydrochloride, xylocalne hydrochloride, benzyl alcohol or phenol; an antiseptic such as benzyl alcohol, phenol, methyl- or propyl-paraben, or chlorobutanol; a buffer such as a sodium salt of citric acid, acetic acid or phosphoric acid; a solubilizing agent such ethanol, propylene glycol or arginine hydrochloride; a stabilizer such as L-cysteine, L-methionine or L-histidine; or an additive such as a tonicity agent.

### 5. Process for Preparing an Aqueous Alkyl Gallate Solution

Alkyl gallates are difficult to prepare into pharmaceutical preparations because they are highly hydrophobic and sparingly soluble in water. The present invention relates to a process for preparing a transparent aqueous solution of alkyl gallates. An alkyl gallate (1 part by weight), a nonionic surfactant (1 to 10 parts by weight) and water (100 to 5000 parts by weight) are mixed with a mixer or by ultrasonication and heated to 30 to 95° C. for dissolution to give a creamy white mixture, which is cooled to room temperature (about 0 to 30° C.), whereby a transparent aqueous solution can be prepared. Examples of the nonionic surfactant include sucrose fatty acid esters, polyoxyethylene castor oil/hardened castor oil, glycerin fatty acid esters, polyethylene macrogol and the like.

### Example 1

Octyl gallate (100 mg), sucrose stearic acid ester (Mitsubishi Chemical Foods Co., Ltd. J1816) (300 mg) and water (100 ml) are mixed with a high speed mixer and heated to about 60 to 70° C. to give a creamy white mixture. This mixture is allowed to stand to room temperature, whereby a transparent aqueous solution is obtained.

### Example 2

To Milli-Q water (about 60 ml) heated to 50 to 70° C., octyl gallate (100 mg) is added, and the mixture is vigorously shaken. After octyl gallate is completely dispersed in water, sucrose fatty acid ester (usually 10 mg/ml, Mitsubishi Chemical Foods Co., Ltd. J1216 (D1216), J1416 (D1416), J1616 (D1616), J1816 (D1816), or the like) (10 to 35 ml) which has been solubilized with Milli-Q water in advance is added thereto, and then stirred, whereby a transparent and colorless aqueous solution is obtained. Then, Milli-Q water is added thereto to make the final volume 100 ml.

### Example 3

To Milli-Q water (100 ml) heated to about 60 to 70° C., propyl gallate (300 to 500 mg) is added, and the mixture is vigorously shaken, whereby a transparent and colorless aqueous solution is obtained.

### Example 4

Octyl gallate (100 mg), polyoxyethylene hardened castor oil (Nikko Chemicals Co., Ltd.; HCO-60) (500 mg), and water (100 ml) are mixed with a high speed mixer and heated to about 60 to 70° C. to give a slightly creamy white mixture. This mixture is allowed to stand to room temperature, whereby a transparent aqueous solution is obtained.

### Example 5

Propyl gallate (500 mg), 5 mM phosphate buffer (KH₂PO₄—Na₂HPO₄) of pH 6.5 and Milli-Q water (100 ml) at 50 to 60° C. are mixed and homogenized with a high speed homogenizer such as Potter-Elvehjem Teflon (registered trademark) glass homogenizer, whereby a transparent and colorless aqueous solution is obtained. The resulting aqueous solution is stored in a brown glass bottle. It is preferred that light is shaded during the preparation of the aqueous solution. The storage is carried out at room temperature or in a refrigerator. When the air contained in the obtained aqueous alkyl gallate solution is replaced with argon gas, He gas, N₂ gas or the like, or an antioxidant is added thereto, the resulting solution can be stored permanently.

### Example 6

To octyl gallate (100 mg) and sucrose stearic acid ester (Mitsubishi Chemical Foods Co., Ltd. J1816) (100 to 300 mg), Milli-Q water (about 50 ml) heated to 60 to 70° C. is added, and the mixture is homogenized with Potter-Elvehjem Teflon (registered trademark) glass homogenizer at a high speed, whereby an aqueous solution which is colorless but slightly turbid is obtained. To the obtained aqueous solution, Milli-Q water is added to make the final volume 100 ml. The storage is carried out at room temperature or in a refrigerator.

### Example 7

To octyl gallate (100 mg), polyethylene glycol (Daiichi Kogyo Pharmaceutical. Co. Ltd., macrogol #6000) (100 to 500 mg) and Milli-Q water (about 50 ml) are added, and the mixture is heated to 40 to 70° C. and stirred to dissolve the ingredients. Then, sucrose stearic acid ester (Mitsubishi Chemical Foods Co., Ltd. J1816) (100 to 300 mg) is added thereto, and the mixture is homogenized with Potter-Elvehjem Teflon (registered trademark) glass homogenizer at a high speed, whereby a completely transparent aqueous solution is obtained. Milli-Q water is added thereto to make the final volume 100 ml. The storage is carried out at room temperature or in a refrigerator.

### Example 8

To Milli-Q water (100 ml) heated to about 70° C., octyl gallate (10 mg) is added, and the mixture is vigorously shaken. After octyl gallate is completely dispersed in water, 1 M arginine hydrochloride is added thereto, whereby a transparent and colorless aqueous solution is obtained. Arginine hydrochloride may be an alkyl arginine hydrochloride such as butyloyl arginine hydrochloride.

### Example 9

### Preparation of Fungicidal, Virucidal and Bactericidal Cocktail

As a fungicidal, virucidal and bactericidal cocktail, the following cocktail was prepared.

### Optimized Formulation (1)

Octyl gallate<200 mg/L (this upper limit is the concentration permitted in quasi-drugs by the Japanese Ministry of Health, Labour and Welfare)
Propyl gallate<2,000 mg/L (this upper limit is the concentration permitted in pharmaceutical additives by the Japanese Ministry of Health, Labour and Welfare)
J1816<2,000 mg/L
Trisodium citrate or disodium hydrogen citrate<4% (w/v)

### KH₂PO₄—Na₂HPO₄<10 mM

Polyethylene glycol
Macrogol #6000<100 mg/L
Antioxidant such as ascorbic acid, sodium ascorbate or vitamin E<1,000 mg/L

### Final pH: 4 to 8

It was found that when the above cocktail containing macrogol #6000 was put on a toothbrush and teeth were brushed with the toothbrush, plaque and tartar could be easily removed.

When the air contained in an aqueous solution of the cocktail obtained in Example 9 is replaced with argon gas, He₂ or N₂ gas or the like, it can be stored permanently.

### Example 10

### Preparation of Fungicidal, Virucidal and Bactericidal Cocktail

As a fungicidal, virucidal and bactericidal cocktail, the following cocktail was prepared.

### Optimized Formulation (2)

Octyl gallate<200 mg/L
Propyl gallate<2,000 mg/L
J1216<600 mg/L
Disodium hydrogen citrate<4% (w/v)
Buffer such as phosphate buffer
Antioxidant (such as sodium ascorbate or vitamin E)

1. A pharmaceutical composition of alkyl gallates which contains alkyl gallates as active ingredients having an anti-fugal, anti-viral or anti-bacterial effect, in which the alkyl group of the alkyl gallate is bound to a galloyl group to form an ester linkage, characterized by comprising the following two members of alkyl gallates:
(A) an alkyl gallate in which the carbon number of the alkyl group is in the range of 5 to 16; and
(B) another alkyl gallate in which the carbon number of the alkyl group is smaller than that of (A).

2. A pharmaceutical composition of alkyl gallates as claimed in claim 1, characterized in that the carbon number of the alkyl group of the alkyl gallate (B) is in the range of 2 to 7.

3. A pharmaceutical composition of alkyl gallates as claimed in claim 1, characterized by further containing (C) at least one member selected from an alkali metal salt, boric acid, sodium borate and an organic salt.

4. A pharmaceutical composition of alkyl gallates as claimed in claim 1, characterized in that it is an aqueous solution in which the alkyl gallates are solubilized by mixing the alkyl gallates with at least one member selected from a nonionic surfactant, polyethylene glycol and arginine or a hydrochloride of a derivative thereof in an aqueous solution or in a pH buffer.

5. A pharmaceutical composition of alkyl gallates as claimed in claim 4, characterized in that it is an aqueous solution in which the alkyl gallates are solubilized by mixing 1 to 10 parts by weight of a nonionic surfactant and 100 to 5000 parts by weight of water based on 1 part by weight of alkyl gallates.

6. A pharmaceutical composition of alkyl gallates as claimed in claim 5, characterized in that it is an aqueous solution in which the alkyl gallates are solubilized by mixing and heating the alkyl gallates at a temperature of 30 to 95° C. followed by cooling to room temperature.

7. A pharmaceutical composition of alkyl gallates as claimed in claim 3, characterized in that which contains the alkyl gallates (A) and (B) as active ingredients having an fungicidal effect.

8. A pharmaceutical composition of alkyl gallates as claimed in claim 3, characterized in that which contains the alkyl gallates (A) and (B) as active ingredients having an virucidal effect.

9. A pharmaceutical composition of alkyl gallates as claimed in claim 8, characterized in that which contains the alkyl gallates (A) and (B) as active ingredients having an anti-influenza viral effect.

10. A pharmaceutical composition of alkyl gallates as claimed in claim 8, characterized in that which contains the alkyl gallates (A) and (B) as active ingredients having an anti-herpes viral effect.

11. A pharmaceutical composition of alkyl gallates as claimed in claim 3, characterized in that which contains the alkyl gallates (A) and (B) as active ingredients having an anti-bacterial effect.

12. A pharmaceutical composition of alkyl gallates as claimed in claim 11, characterized in that which contains the alkyl gallates (A) and (B) as active ingredients having an anti-MRSA effect.

13. A pharmaceutical composition of alkyl gallates as claimed in claim 12, characterized in that which contains the alkyl gallates (A) and (B) as active ingredients having an anti-MSSA effect.

14. A pharmaceutical composition of alkyl gallates as claimed in claim 12, characterized in that the composition is a β-lactam activity enhancing agent.

15. A pharmaceutical composition of alkyl gallates as claimed in claim 14, characterized in that the β-lactam is oxacillin.

16. A pharmaceutical composition of alkyl gallates as claimed in claim 7, characterized in that the alkyl gallate (A) is octyl gallate, and the alkyl gallate (B) is propyl gallate.

17. A pharmaceutical composition of alkyl gallates as claimed in claim 7, characterized in that the alkyl gallate (A) is octyl gallate, and the alkyl gallate (B) is isoamyl gallate.

18. A pharmaceutical composition of alkyl gallates as claimed in claim 7, characterized in that the alkyl gallate (A) is octyl gallate, the alkyl gallate (B) is propyl gallate, and (C) is disodium hydrogen citrate or trisodium citrate.
